(19) United States
(12) Patent Application Publication
(10) Pub. No.: US 2006/0257381 A1
(43) Pub. Date: Nov. 16, 2006

(54) Method for transplanting lymphohematopoietic cells into mammal

(76) Inventors: Keiya Ozawa, Tochigi (JP); Yutaka Hanazono, Tochigi (JP); Kyoji Ueda, Kyoto (JP); Yasuji Ueda, Ibaraki (JP); Mamoru Hasegawa, Ibaraki (JP)

Correspondence Address: CLARK & ELBING LLP, 101 FEDERAL STREET, BOSTON, MA, 02110, US

(21) Appl. No.: 10/562,322

(22) PCT Filed: Jun. 25, 2004
(86) PCT No.: PCT/JP04/09370

§ 371 Date: May 9, 2006

Related U.S. Application Data
(60) Provisional application No. 60/483,267, filed on Jun. 26, 2003.

(51) Int. Cl. A61K 48/00; C12N 5/08

(52) U.S. Cl. 424/93.21; 435/372; 514/44

(57) ABSTRACT

The present invention provides a method for transplanting lymphohematopoietic cells into a mammal, which comprises the step of injecting cells into a bone marrow cavity, and wherein the cells have an exogenous gene encoding a receptor that induces cell proliferation in response to ligand binding. By combining intra-bone marrow transplantation (iBMT) and selective amplifier gene (SAG), marrow conditioning before the injection of the cells can be omitted. The present invention further provides a bone marrow transplant and a kit for transplanting lymphohematopoietic cells into mammals. Furthermore, the invention provides an SAG particularly suitable for such transplantation.

The present application is related to U.S. Ser. No. 60/483,357, filed Jun. 27, 2003, which is incorporated herein by reference.

### TECHNICAL FIELD

The present invention relates to the field of genetic engineering, particularly to the field of gene therapy. Specifically, the present invention relates to a method for transplanting lymphohematopoietic cells into mammals. Furthermore, the present invention relates to a bone marrow transplant and a kit for transplanting lymphohematopoietic cells into mammals. Moreover, the invention relates to a gene encoding a fusion protein adapted for such transplantation.

### BACKGROUND ART

Although a few hematopoietic stem cell (HSC) gene therapy trials have proven successful (Cavazzana-Calvo et al., Science 2000, 288: 669-72; Aiuti et al., Science 2002, 296: 2410-3), one of the major obstacles associated with HSC gene therapy is the low efficiency of gene transfer into human HSCs with retroviral vectors (Dunbar et al., Blood 1995, 85: 3048-57). Therefore, methods enabling selection of transduced cells still contribute to the clinical application of HSC gene therapy. Since recent studies revealed that hematopoietic stem cells have pluripotency to differentiate into type of cells other than blood cells, such as endothelial cells or skeletal muscle myoblasts (Rafii et al., Blood 1994, 84: 10-19; Ferrari et al., Science 1998, 279: 528-530), the HSC gene therapy will be applicable for other disorders than blood diseases.

A strategy of in vivo selection of transduced hematopoietic cells utilizes a drug-resistance gene, such as multidrug resistance 1 (MDR-1) gene (Sorrentino et al., Science 1992, 257: 99-103), mutant dihydrofolate reductase (DHFR) gene (Allay et al., Nat Med 1998, 4: 1136-43) or DNA alkyltransferase gene (Davis et al., Cancer Res 1997, 57: 5093-9; Raggs et al., Cancer Res 2000, 60: 5187-95; Sawai et al., Mol Ther 2001, 3: 78-87). Although the strategy has been successful in mice, it has been proven less effective in human subjects and nonhuman primates (Hanania et al., Proc Natl Acad Sci USA 1996, 93: 15346-51; Moscow et al., Blood 1999, 94: 52-61; Abonour et al., Nat Med 2000, 6: 652-8). Furthermore, the administration of agents, such as taxol (for MDR-1 selection) or methotrexate (for DHFR selection), required for this method is highly toxic.

Another strategy of in vivo positive selection of transduced cells confers a direct proliferation advantage on gene-modified cells relative to their untransduced counterparts. The present inventors developed a chimeric gene dubbed “selective amplifier gene (SAG)”, which encodes a chimeric receptor of the granulocyte colony-stimulating factor (G-CSF) receptor (GCR) and the hormone-binding domain of the estrogen or tamoxifen receptor (Ito et al., Blood 1997, 90: 3884-92; Matsuda et al., Gene Ther 1999, 6: 1038-44; Xu et al., J Gene. Med 1999, 1: 236-44; Nagashima et al., Biochem Biophys Res Commun 2003, 303: 170-6; Kume et al., J Gene Med 2003, 5: 175-81; Hanazono et al., Gene Ther 2002, 9: 1055-64). The GCR moiety is a growth signal generator and the estrogen receptor (ER) moiety a molecular switch that regulates (turns on or off) the growth signal generated by the GCR.

Cytokine receptors generate the growth signal through ligand-induced dimerization. Unligated cytokine receptor dimers exist in a conformation that prevents signal generation but undergoes a ligand-induced conformation change that allows signal generation (Livnah et al., Science 1999, 283: 987-90; Remy et al., Science 1999, 283: 990-3). Thus, dimerization is necessary, however, not sufficient for optimal signal generation.

In vivo expansion of gene-modified cells is a promising approach in the field of HSC gene therapy. Such method would circumvent low gene transfer efficiency into HSCs, which is one of the current limitations of the promising technology. Previous papers documented that, without marrow conditioning, very low levels (much less than 0.1%) of cells were marked (or corrected) after CD34⁺ cell gene therapy of chronic granulomatous disease and Gaucher's disease (Malech et al., Proc Natl Acad Sci USA 1997, 94: 12133-8; Dunbar et al., Hum Gene Ther 1998, 9: 2629-40). Furthermore, the ability to expand genetically modified cells in vivo would circumvent another major problem of HSC gene therapy, i.e., the need of myeloablative conditioning unless gene-modified cells have clear growth advantage (Cavazzana-Calvo et al., Science 2000, 288: 669-72). Current myeloablative conditioning regimens are associated with high systemic toxicity, and potential damage to marrow stroma possibly resulting in impaired engraftment (Plett et al., Blood 2002, 100: 3545-52). Through the in vivo selection method using a drug resistance gene, engraftment of transduced cells at low levels may allow successful expansion to clinically relevant levels even without marrow conditioning. However, such method requires administration of cytotoxic agents for the selection (Bowman et al., Mol Ther 2003, 8: 42-50).

Alternatively, the SAG encoding GCR as a growth-signal generator and the hormone-binding domain of a steroid receptor (estrogen or tamoxifen receptor) as a molecular switch, previously developed by the present inventors accomplish in vivo and in vitro steroid-dependent expansion of hematopoietic cells retrovirally transduced with the gene in mice and nonhuman primates. However, this SAG failed to induce the increase of transduced cells in some animals. The fusion protein of GCR and estrogen receptor was revealed to more efficiently respond to G-CSF than to estrogen (Ito et al., Blood 1997, 90: 3884-92). Therefore, the estrogen-mediated dimerization of the chimeric molecule may be less efficient than the natural ligand (G-CSF)-mediated dimerization, and thus, the use of steroid receptor may have attenuated the potency of SAG. Furthermore, the administration of steroids, such as estrogen and tamoxifen, may cause side effect.

Similar to the chimeric receptors constructed by the present inventors, a cell growth switch, a cytokine receptor-FK506 binding protein (FKBP) fusion gene, has been also developed by Blau et al. that confers inducible proliferation to transduced cells (Blau et al., Proc Natl Acad Sci USA 1997, 94: 3076-81; Richard et al., Blood 2000, 95: 430-6). In the system of Blau et al., the cytokine receptor signal is turned on by the treatment with a synthetic dimerizer FK1012 or derivatives thereof. However, it remains unclear whether their chimeric protein allows effective ligand-induced conformation change.

It has recently been reported that bone marrow cells can efficiently engraft mice without marrow conditioning when directly transplanted into the bone marrow cavity (intra-bone marrow transplantation; iBMT) (Zhong et al., Blood 2002, 100: 3521-6; Nakamura et al., Stem Cells 2004, 22: 125-34). Using the iBMT method, human cord blood cells are also able to engraft efficiently in bone marrow of sublethally irradiated immunodeficient mice (Wang et al., Blood 2003, 101: 2924-31; Mazurier et al., Nat Med 2003, 9: 959-63; Yahata et al., Blood 2003, 101: 2905-13). Although the iBMT method has been successful in mice, the efficacy in primates remains to be examined.

### DISCLOSURE OF THE INVENTION

The words “a”, “an” and “the” as used herein mean “at least one” unless otherwise specifically indicated. Unless otherwise defined, all technical and scientific terms used herein have the same meaning as commonly understood by one of ordinary skill in the art to which this invention belongs.

1. New Generation SAG

An objective of the present invention is to provide an SAG that encodes a more stable and compatible fusion protein that can be simulated with a factor without causing serious adverse effect than the hitherto reported SAG, which comprised the hormone-binding domain of a steroid receptor.

In the present invention, the inventors have developed a new generation SAG that encodes an erythropoietin (EPO) receptor (EPOR) in place of the steroid receptor. Specifically, the ligand-binding domain of EPOR is used as the molecular switch to regulate (turns on or off) the growth signal generated by the cytoplasmic domain of the cytokine receptor. Thus, the present invention provides a gene encoding a fusion protein comprising (a) a ligand-binding domain of EPOR, and (b) a growth signal generator that imparts proliferation activity to a cell upon the binding of a ligand.

Similar to GCR, EPOR is a member of the cytokine receptor superfamily (Bazan, Proc Natl Acad Sci USA 1990, 87: 6934-8). Therefore, a fusion protein of EPOR and a growth signal generator derived from a cytokine receptor (e.g., GCR) should be more stable and compatible than that of a hormone receptor and GCR. Furthermore, EPOR is not expressed on immature hematopoietic cells and thus are a suitable selective switch for these cells (Suzanne et al., Proc Natl Acad Sci USA 1996, 93: 9402-7). Moreover, recombinant human EPO has been widely used in clinical application and is known to be repeatedly applicable to human subjects without causing serious adverse effects (Brandt et al., Pediatr Nephrol 1999, 13: 143-7; Itri, Semin Oncol 2002, 29: 81-7). Thus, the present new generation SAG utilizing EPO as the ligand is expected to be a promising tool for in vivo expansion of gene-modified cells.

The extracellular region, preferably the ligand-binding domain of EPOR is used for the fusion protein of the present invention. EPOR may be derived from any species; however, for use in human gene therapy it is particularly preferred to use the human EPOR.

In the clinical setting, even if the expansion of gene-modified cells is transient, patients can expect therapeutic effects by EPO administration when necessary such as infection events in patients with chronic granulomatous disease. EPO is a safe drug and can be administered repeatedly with minimal adverse effects. Polycythemia was the only side effect observed in the present study, and polycythemia is manageable by periodic phlebotomy. Therapeutic effects might also be expected from continuously elevated levels of endogenous EPO such as in patients with thalassemia for instance. When anemia is ameliorated and endogenous EPO levels return to physiological levels, then the positive selection system is “automatically” turned off, making this a convenient system in such disorders.

Although this “leave it to patient” system is convenient, a safety concern may be raised regarding leukemogenesis (Hacein-Bey-Abina et al., Science 2003, 302: 415-9). The SAG proliferation signal that is persistently turned on in vivo by endogenous EPO could trigger a secondary event in addition to possible retroviral insertional mutagenesis, although physiological levels of EPO will not induce a significant proliferation response of SAG (Nagashima et al., J Gene Med 2004, 6: 22-31).

Therefore, an SAG encoding the ligand-binding domain of an EPOR that does not bind to endogenous EPO but to EPO-mimetic peptides would be more preferred and are included in the present invention. Herein, EPO-mimetic peptides include modified or mutant EPO, such as erythropoiesis stimulating protein (NESP) developed by Wrighton et al. or Macdougall (Wrighton et al., Science 1996, 273: 458-64; Macdougall, Semin Nephrol 2000, 20: 375-81). Such EPOR binding to EPO-mimetic peptides can be obtained by modifying native EPOR by site-directed mutagenesis and such, and then determining their binding ability to endogenous EPO and EPO-mimetic peptides.

The SAG of the present invention encodes a fusion protein that comprises a growth signal generator in addition to the ligand-binding domain of EPOR. The growth signal generator is not restricted in any way so long as it imparts proliferation activity to a cell upon the binding of a ligand to the ligand-binding domain of EPOR. Thus, the whole or a part of the cytoplasmic domain of a cytokine receptor may be used in the present invention as a growth signal generator. Furthermore, the cytoplasmic domain encoded in the SAG of the present invention may be derived from any cytokine receptor so long as it imparts the proliferation activity; however, preferred cytoplasmic domains include those belonging to the cytokine receptor family encompassing GCR and c-Mpl, and the thyrosine kinase receptor family (e.g., c-kit, flk2/flt3, etc.).

According to the invention, as the new generation SAGs, two EPO-driven SAGs were constructed, i.e., EPORGCR and EPORMpl containing GCR and c-Mpl, respectively. These SAGs were shown to induce more rapid and potent proliferation of Ba/F3 cells than the steroid-driven SAGs. The results reported herein indicate that SAGs utilizing EPOR as a molecular switch is more efficient for hematopoietic cell proliferation than that utilizing the steroid (or tamoxifen) receptor despite the inclusion of the same signal generator (GCR) in the SAGs. The EPO-driven SAG might have allowed more effective ligand-induced conformation change than the steroid-driven SAG. Furthermore, the c-Mpl signal was shown to much efficiently expand clonogenic progenitor cells (colony-forming units; CFU) compared to the EPOR or GCR signal. In addition, the cells expanded by c-Mpl signal showed the most balanced expression of myeloid, erythroid and megakaryocyte markers. Taken together, the intracellular signal from c-Mpl may be suitable for reliable expansion of immature hematopoietic cells. Thus, an SAG that encodes a fusion protein comprising the ligand-binding domain of EPOR and the cytoplasmic region of c-Mpl is a particularly preferred example of the present invention.

c-Mpl is the receptor of thrombopoietin (TPO). c-Mpl is expressed on very immature hematopoietic cells and actually stimulates the growth of these cells (Borge et al., Blood 1997, 90: 2282-92; Solar et al., Blood 1998, 92: 4-10; Kimura et al., Proc Natl Acad Sci USA 1998, 95: 1195-200; Kaushansky, Leukemia 2002, 16: 738-9). In fact, the cytoplasmic fragment of c-Mpl has been used for cell expansion (Nagashima et al., Biochem Biophys Res Commun 2003, 303: 170-6; Gurney et al., Proc Natl Acad Sci USA 1995, 92: 5292-6). c-Mpl signal has also been demonstrated to efficiently support the growth of transduced murine bone marrow cells (Zeng et al., Blood 2001, 98: 328-34).

SAGs of the present invention can be constructed by conventional gene engineering techniques. Specifically, DNAs encoding the ligand-binding domain and the growth signal generator are linked together to be expressed as one polypeptide. More specifically, the region encoding the intracellular domain of EPOR in the EPOR encoding gene may be replaced with a portion encoding the cytoplasmic region of a cytokine receptor as described in Example 1 (see also FIG. 1).

Preferably, the present SAG is virally transduced into host cells to express the fusion protein in vivo. Any viral vector may be used for introducing the gene into a host cell; however, it is a mammalian cell-infecting viral vector that is less toxic to host cells and achieves a high expression level of a transgene. Viral vectors that can be used for expressing the fusion protein of the present invention include those recombinant viral vectors constructed by gene manipulation of adenovirus, adeno-associated virus, herpes simplex virus, retrovirus, lentivirus, Semliki forest virus, Sindvis virus, vaccinia virus, fowl pox virus and Sendai virus. The recombinant viral vectors may be generated by reconstituting virus particles through the expression of recombinant virus cDNA in host cells.

The recombinant viral vectors may be prepared according to methods known to those skilled in the art. For example, an adenoviral vector that is most frequently used for gene therapy can be constructed following the method of Saito et al. (Miyakae et al., Proc Natl Acad Sci USA 1996, 93: 1320-4; Kanegae et al., “Biomanual Series 4-Gene Transfer and Expression, Methods of Analysis (in Japanese)” 1994, 43-58, Yodosha). Alternatively, retroviral vectors (Wakimoto et al., Protein Nucleic Acid and Enzyme 1995, 40: 2508-13) and adeno-associated viral vectors (Tamaki et al., Protein Nucleic Acid and Enzyme 1995, 40: 2532-8) may also be used. Methods to efficiently produce these vectors are known in the art.

In the interest of producing other vectors that can be used for gene transfer into mammalian cells, Published Japanese Translation of International Publication No. Hei 6-502069, Examined Published Japanese Patent Application No. (JP-B) Hei 6-95937 and JP-B Hei 6-71429 disclose detailed methods for producing recombinant vaccinia viruses. Furthermore, JP-B Hei 6-34727 and Published Japanese Translation of International Publication No. Hei 6-505626 disclose methods for producing recombinant papilloma viruses. Moreover, Unexamined Published Japanese Patent Application No. (JP-A) Hei 5-308975 discloses a method for producing recombinant adeno-associated virus, and Published Japanese Translation of International Publication No. Hei 6-508039 a method for producing recombinant adeno virus. All of these methods can be utilized in the transduction of cells by the present SAG.

The envelope protein of a viral vector may contain a protein other than the envelope protein of the original vector genome. For example, viral vectors having desired envelope proteins can be produced by intracellularly expressing envelope protein other than that encoded by the virus genome during viral reconstitution. There is no limitation on such proteins and include envelope proteins of other viruses such as the G protein (VSV-G) of the vesicular stomatitis virus (VSV). Thus, pseudo-type viral vectors that have an envelope protein derived from a virus different from the original virus may be used for the expression of the present SAG.

The viral vector may also comprise, for example, on the viral envelope surface, proteins capable of adhering to particular cells, such as adhesion factors, ligands and receptors or chimeric proteins on the outer surface and viral envelope-derived polypeptides inside the virus. Such adhering proteins enable the production of a vector targeting a particular tissue. These proteins may be encoded by the virus genome itself or supplied at the time of virus reconstitution through expression of genes other than virus genome (for example, genes derived from another expression vector or host cell chromosome)

The virus genes contained in the viral vector may be altered, for example, to reduce antigenicity of the virus protein derived from the vector, or enhance RNA transcription efficiency or replication efficiency.

The SAG of the present invention is inserted into the viral vector DNA. For example, when a Sendai virus vector is used, a sequence comprising nucleotides of multiples of six is desirably inserted between the transcription end sequence (E) and the transcription start sequence (S) (J Virol 1993, 67 (8): 4822-30). An exogenous gene can be inserted upstream and/or downstream of each of the virus genes (NP, P, M, F, HN and L genes) in a viral vector. In order not to interfere with the expression of upstream and downstream genes, an E-I-S sequence (transcription end sequence-intervening sequence-transcription start sequence) or a portion thereof may be suitably placed upstream or downstream of an exogenous gene so that the unit of E-I-S sequence is located between each gene. Alternatively, an exogenous gene can be inserted via internal ribosome entry site (IRES) sequence.

The fusion protein of the present invention introduced into a host cell is expressed on the cell surface to allow binding of a ligand to the ligand binding domain, and finally impart proliferation activity to the cell. The expression level of inserted SAG can be regulated by the type of transcription start sequence that is attached to the upstream of the gene (WO 01/18223). It also can be regulated by the position of insertion and the sequence surrounding the gene. For example, in the Sendai virus, the closer to the 3′-terminus of the negative strand RNA of the virus genome (the closer to the NP gene in the gene arrangement on the wild-type virus genome) the insertion position is, the higher the expression level of the inserted gene will be. Conversely, the closer to the 5′-terminus of the negative strand RNA (the closer to the L gene in the gene arrangement on the wild-type virus genome) the insertion position is, the lower the expression level of the inserted gene will be. Thus, the insertion position of an exogenous gene can be properly adjusted to obtain a desired expression level of the gene or optimize the combination of the insert with the virus genes surrounding it.

To help easy insertion of an exogenous gene, a cloning site may be designed at the position of insertion in the vector DNA encoding the genome. For example, the cloning site may be the recognition sequence of a restriction enzyme. The cloning site may be a multicloning site that contains recognition sequences for multiple restrictionenzymes. The viral vector may have other exogenous genes at positions other than that used for the insertion of the present SAG. Such exogenous gene may be, without limitation, a marker gene or another gene.

The SAG of the present invention can be inserted into a viral vector using such cloning sites. Then, the recombinant viral vector containing SAG is bound to an appropriate transcription promoter and the resultant DNA is transcribed in vitro or intracellularly to reconstitute the virus. The reconstitution of a virus from a viral vector DNA can be performed according to known methods (WO 97/16539; WO 97/16538; Durbin et al., Virol 1997, 235: 232-32; Whelan et al., Proc Natl Acad Sci USA 1995, 92: 8388-92; Schnell et al., EMBO J. 1994, 13: 4195-203; Radecke et al., EMBO J. 1995, 14: 5773-84; Lawson et al., Proc Natl Acad Sci USA 1995, 92: 4477-81; Gacin et al., EMBO J. 1995, 14: 6087-94; Kato et al., Genes Cells 1996, 1: 569-79; Baron and Barrett, J Virology 1997, 71: 1265-71; Bridgen and Elliott, Proc Natl Acad Sci USA 1996, 93: 15400-4). These methods enable the reconstitution of desirable Paramyxovirus vectors including the parainfluenza virus, vesicular stomatitis virus, rabies virus, measles virus, rinderpest virus and Sendai virus vectors and the other (−) strand RNA viral vectors from DNA.

Methods for introducing vector DNA into cells include: (1) a method for forming DNA precipitates that can be incorporated into desired cells; (2) a method for making a complex that comprises positively charged DNA that is suitable for being incorporated into desired cells and that has low cytotoxicity; and (3) a method for instantaneously opening a pore large enough for DNA to pass through the desired plasma membrane using an electrical pulse.

For (1), transfection using calcium phosphate can be used. In this method, DNA incorporated by cells is taken up into phagocytic vesicles, but it is known that a sufficient amount of DNA is also taken up into the nucleus (Graham and van Der Eb, Virol 1973, 52: 456; Wigler and Silverstein, Cell 1977, 11: 223). Chen and Okayama studied the optimization of the transfer technology and reported (1) that maximal efficiency is obtained when cells and precipitates are incubated under 2% to 4% CO₂ at 35° C. for 15 hr to 24 hr; (2) that circular DNA has higher activity than linear DNA; and (3) that the optimal precipitates, are formed when the DNA concentration in the mixed solution is 20 μg/ml to 30 μg/ml (Chen and Okayama, Mol Cell Biol 1987, 7: 2745).

A variety of transfection reagents can be used in (2) including DOTMA (Boehringer), Superfect (QIAGEN #301305), DOTAP, DOPE and DOSPER (Boehringer #1811169). This method is suitable for transient transfection. More classically, a transfection method wherein DEAE-dextran (Sigma #D-9885 M. W. 5×10⁵) is mixed with DNA at a desired concentration ratio is known. Because most complexes are degraded in the endosome, chloroquine may be added to enhance the transfection efficiency (Calos, Proc Natl Acad Sci USA 1983, 80: 3015).

The method of (3), called electroporation, can be used for any kind of cells, thus can be more broadly applied than the methods (1) and (2). The transfection efficiency can be maximized by optimizing the duration of pulse currents, the form of pulse, the strength of the electrical field (gap between electrodes, and voltage), conductivity of buffer, DNA concentration and cell density.

Host cells for viral reconstitution are not limited to any special types of cells as long as the viral vector can be reconstituted in the cells. For example, in order to reconstitute a SeV vector and the like, host cells including monkey kidney-derived cells such as LLC-MK2 cells and CV-1 cells, cultured cell lines such as BHK cells derived from a hamster kidney, and human-derived cells may be used. By expressing appropriate envelope proteins in these cells, infectious viral particles that include the proteins in its envelope can be obtained. Furthermore, to obtain a large quantity of the viral vector, embryonated chicken eggs may be infected with viral vectors obtained from the above host cells and the vectors can be amplified. The method of producing viral vectors using chicken eggs has been established (Advanced protocols in neuroscience study III, Molecular physiology in neuroscience, Ed. by Nakanishi et al., 1993, 153-172, Kouseisha, Osaka). Specifically, for example, fertilized eggs are incubated for 9 days to 12 days at 37° C. to 38° C. in an incubator to grow the embryos. Viral vectors are inoculated, into the allantoic cavity, and eggs are further incubated for several days to propagate the vectors. Conditions such as the duration of incubation may vary depending on the type of recombinant virus used. Then, the allantoic fluids containing viruses are recovered. The viral vector is separated and purified from the allantoic fluid sample according to standard methods (see, Tashiro, “Protocols in virus experiments.”, Ed. by Nagai and Ishihama, 1995, 68-73, MEDICAL VIEW).

The collected virus may be purified substantially pure. The purification can be carried out by known purification/separation methods including filtration, centrifugation and column purification, or combinations thereof. The phrase “substantially pure” means that the virus is the major portion of a sample where it is present as a component. Typically, a sample can be confirmed to be a substantially pure viral vector when proteins derived from the viral vector occupies 10% or more, preferably 20% or more, more preferably 50% or more, more preferably 70% or more, more preferably 80% or more, and even more preferably 90% or more, of the total proteins (but excluding proteins added as carriers or stabilizers) in the sample. Specific examples of purification methods for Paramyxovirus include methods using cellulose sulfate ester or cross-linked polysaccharide sulfate ester (JP-B Sho 62-30752; JP-B Sho 62-33879; JP-B Sho 62-30753), and those including adsorption of the virus with fucose sulfuric acid-containing polysaccharide and/or its degradation product (WO 97/32010).

A viral vector containing SAG of the present invention is used to transduce cells that can be utilized for gene therapy. Preferred cells include lymphohematopoietic cells, particularly puluripotent stem cells. For example, by selecting CD34⁺ cells from peripheral blood or bone marrow cells, cells preferred for introducing the present SAG can be obtained. To use the cells in gene therapy, it is particularly preferred to obtain the cells from peripheral blood or bone marrow cells collected from the subject to be treated.

The cells transduced by a viral vector containing the SAG of the present invention are then introduced into the subject to be treated. The transplantation of cells may be achieved by injection into the blood vessel or bone marrow, i.e., intravenous transplantation or intra-bone marrow transplantation (iBMT). iBMT is a particularly preferred method to attain a high gene marking level.

To induce proliferation of the transplanted cells, a ligand (EPO or EPO-mimetic peptide) of the transduced SAG is administered to the patient. The ligand may be administered by intravenous or subcutaneous injection, for example, at a dose of 200 IU/kg once to few times daily. However, the present invention is not restricted to this method, and the ligand may be administered via appropriate routes at a suitable dose that achieves the transmission of the signal (proliferation activity) from the introduced fusion protein on the cell surface.

2. Transplantation Method

The very low level of marked (or corrected) cells after CD34⁺ cell gene therapy of chronic granulomatous disease and Gaucher disease (Malech et al., Proc Natl Acad Sci USA 1997, 94: 12133-8; Dunbar et al., Hum Gene Ther 1998, 9: 2629-40) has formed the foundation for the contention that myeloablation (or at least conditioning of reduced intensity) is required for successful engraftment of transplanted, genetically modified cells. Successful engraftment of genetically modified HSCs without toxic conditioning is a desired goal for HSC gene therapy.

Therefore, the present inventors examined the combination of iBMT and in vivo expansion by an SAG in nonhuman primate model. A chimeric gene consisting of the EPOR as a molecular switch and c-Mpl gene as a signal generator was used as the SAG. Cynomolgus CD34⁺ cells were retrovirally transduced with or without-SAG and returned into the femur and humerus following irrigation with saline without prior conditioning. After iBMT without SAG, 2 to 30% of colony-forming cells were gene-marked over one year. The marking levels in the peripheral blood, however, remained low (<0.1%). These results indicate that transplanted cells can engraft without conditioning after iBMT with limited expansion in vivo. On the other hand, after iBMT with SAG, the peripheral marking levels increased more than 20-fold (up to 8 to 9%) in response to EPO even after one year from transplantation. The increase was EPO-dependent, multilineage, polyclonal and repeatable. These results suggest that the combination of iBMT and SAG allows efficient in vivo gene transduction without marrow conditioning.

Thus, the present invention provides a method for transplanting lymphohematopoietic cells into a mammal, which comprises the step of injecting cells into a bone marrow cavity, and wherein the cells have an exogenous gene encoding a receptor that induces cell proliferation in response to ligand binding. By combining iBMT and SAG, marrow conditioning before the injection of the cells can be omitted.

iBMT can be performed as described in Example 2 under the item of “(4) Intra-bone marrow transplantation”. Furthermore, iBMT can be performed according to reported methods (e.g., Zhong et al., Blood 2002, 100: 3521-6; Nakamura et al., Stem Cells 2004, 22: 125-34; Wang et al., Blood 2003, 101: 2924-31; Mazurier et al., Nat Med 2003, 9: 959-63; Yahata et al., Blood 2003, 101: 2905-13). Specifically, needles are inserted into both ends of the bone, and gene-modified cell-containing solution is injected into the marrow cavity. The injection should be performed without inflicting extra-pressure to the marrow cavity. The physical elimination of endogenous marrow with saline before injection might increase gene marking. Thus, preferably, the bone cavity is washed with, for example, heparin-added saline before iBMT. The cells for transplantation are preferably suspended in saline. The saline may contain other ingredients so long as it does not inhibit the transplantation of the cells, the expression of the receptor, ligand binding, proliferation of the cells and so on.

According to the present invention, the marrow cavity of one or more of femurs, humeri, iliac bones and such may be the target of transplantation. In the current study, the marrow of four proximal limb bones (femurs and humeri) was replaced with transplanted cells. When other bones such as the iliac bone (that contains more marrow) are similarly used for iBMT, even higher in vivo marking level may be achieved. Thus, to attain a high in vivo marking level, it is preferred to transplant the cells into as many bone marrow cavities as possible.

The present method may be applied to any mammal; however, Primates are particularly preferred. For example, the present method may be applied for gene therapy of Primates belonging to Prosimii and Anthropoidea, including human.

The lymphohematopoietic cells used in the present transplantation method have an exogenous gene encoding a receptor that induces cell proliferation in response to ligand binding. Preferred lymphohematopoietic cells include pluripotent stem cells. For example, by selecting CD34⁺ cells from peripheral blood or bone marrow cells, cells suitably used for the present method can be obtained. To use the cells in gene therapy, it is particularly preferred to obtain cells from peripheral blood or bone marrow cells collected from the subject to be treated.

The exogenous gene is suitably introduced into the cells using a viral vector. Any viral vector may be used for introducing the gene into a host cell; however, it is a mammalian cell-infecting viral vector that is less toxic to host cells and achieves a high expression level of a transgene. Viral vectors that can be used for expressing the fusion protein of the present invention include those recombinant viral vectors constructed by gene manipulation of adenovirus, adeno-associated virus, herpes simplex virus, retrovirus, lentivirus, Semliki forest virus, Sindvis virus, vaccinia virus, fowl pox virus and Sendai virus. The recombinant viral vectors may be generated by reconstituting virus particles through the expression of recombinant virus cDNA in host cells. Methods for preparing viral vectors are well known in the art and any method may be utilized for the present invention. See supra, under the item of “1. New generation SAG”.

The exogenous gene of the present invention encodes a receptor that induces cell proliferation in response to ligand binding. Such endogenous genes are exemplified by those encoding receptors that comprise a growth signal generator, such as cytokine receptors, including TPO receptor (c-Mpl) and G-CSF receptor (GCR). In the present invention, to regulate in vivo proliferation of the transplanted cells, it is preferred to use genes encoding artificial chimeric proteins that comprise a growth signal generator and a ligand-binding domain.

Cytokine receptors generate the growth signal through ligand-induced dimerization to induce cell proliferation of the cell. Therefore, it is preferred to use a chimeric protein that comprises (a) an extracellular domain of a receptor that dimerizes the chimeric protein in response to ligand binding, and (b) a growth signal generator that induces cell proliferation in response to the dimerization.

An “extracellular domain of a receptor that dimerizes the chimeric protein in response to ligand binding” can be exemplified by hormone-binding domains (e.g., estrogen or tamoxifen receptor) used in the previously reported SAG (Ito et al., Blood 1997, 90: 3884-92; Matsuda et al., Gene Ther 1999, 6: 1038-44; Xu et al., J Gene Med 1999, 1: 236-44; Nagashima et al., Biochem Biophys Res Commun 2003, 303: 170-6; Kume et al., J Gene Med 2003, 5: 175-81; Hanazono et al., Gene Ther 2002, 9: 1055-64), and the ligand-binding domain of EPOR of above-described SAG of the present invention. The ligand-binding domain, i.e., the extracellular domain of EPOR is particularly preferred for the method of the present invention.

The “growth signal generators” of the present invention are not restricted in any way so long as they induce cell proliferation of lymphohematopoietic cells in response to the binding of a ligand to the ligand-binding domain or the dimerization of the chimeric protein. Such growth signal generators include the cytoplasmic domain of a hematopoietic cytokine receptor, such as c-Mpl or GCR. However, the cytoplasmic domain of other cytokine receptors may also be used in the present invention, and those belonging to the cytokine receptor family encompassing GCR and c-Mpl as well as the tyrosine kinase receptor family (e.g., c-kit, flk2/flt3, etc.) can also be used.

The endogenous gene used for the present method can be constructed as described above under the item of “1. New generation SAG”. An SAG that encodes a fusion protein comprising the ligand-binding domain of EPOR and the cytoplasmic region of c-Mpl is a particularly preferred example of the exogenous gene used in the present invention.

The lymphohematopoietic cell transplanted according to the present method preferably comprises a vector having a therapeutic gene in addition to the exogenous gene encoding a receptor. Herein, the phrase “therapeutic gene” refers to a gene that may be used for gene therapy. Hitherto, the object of gene therapy is to introduce a normal gene in place of a defective gene in a subject to let the normal gene produce a normal protein that ameliorates the symptoms of the subject. Thus, genes that encode such normal proteins ameliorating the symptoms of the subject upon transduction into the lymphohematopoietic cell can be used as the therapeutic gene of the present invention. For example, genes used in the gene therapy of chronic granulomatous disease, Gaucher's disease and Fanconi anemia (Malech et al., Proc Natl Acad Sci USA 1997, 94: 12133-8; Dunbar et al., Hum Gene Ther 1998, 9: 2629-40; Walsh et al., J. Investing Med. 1995, 43:379-85) are preferred examples of the therapeutic gene of the present invention

Since the proliferation of the cells transplanted according to the present method are induced in response to ligand binding, the present method may include the step of administering a ligand of the receptor encoded by the exogenous gene into the subject mammal. Any ligand may be used so long as it binds to the receptor and induces dimerization of the receptor to finally induce proliferation of the cell. For example, when the extracellular domain of EPOR or a mutant thereof is used as the ligand-binding domain of the receptor, EPO or EPO-mimetic peptide may be administered to the subject as a ligand.

The ligand may be administered by intravenous or subcutaneous injection, for example, at a dose of 200 IU/kg once to few times daily. However, the present invention is not restricted to this method, and the ligand may be administered via appropriate routes at a suitable dose that achieves the transmission of the signal (proliferation activity) from the receptor on the cell surface,

3. Bone Marrow Transplant

According to the present invention, the combination of iBMT and in vivo expansion by an SAG was demonstrated preferable to achieve high marking levels for a long period in the peripheral blood after transplantation of cells without marrow conditioning. This indicates that SAG containing cells are particularly suited for bone marrow transplantation. Thus, the present invention provides a bone marrow transplant that comprises lymphohematopoietic cells having an exogenous gene encoding a receptor that induces cell proliferation in response to ligand binding. This bone marrow transplant is preferably used in iBMT.

Preferred lymphohematopoietic cells to be used in the present bone marrow transplant include pluripotent stem cells. For example, by selecting CD34⁺ cells from peripheral blood or bone marrow cells, cells suitably used for the present transplant can be obtained. It is particularly preferred to obtain cells from peripheral blood or bone marrow cells collected from the subject to be treated. The cells are derived from any mammal; however, Primates are preferred. For example, Primates belonging to Prosimii and Anthropoidea, including human are particularly preferred.

The exogenous gene is suitably introduced into the cell using a viral vector. Any viral vector may be used for introducing the gene into a host cell; however, it is a mammalian cell-infecting viral vector that is less toxic to host cells and achieves a high expression level of a transgene. Viral vectors that can be used for expressing the fusion protein of the present invention include those recombinant viral vectors constructed by gene manipulation of adenovirus, adeno-associated virus, herpes simplex virus, retrovirus, lentivirus, Semliki forest virus, Sindvis virus, vaccinia virus, fowl pox virus and Sendai virus. The recombinant viral vectors may be generated by reconstituting virus particles through the expression of recombinant virus cDNA in host cells. Methods for preparing viral vectors are well known in the art and any method may be utilized for the present invention. See supra, under the item of “1. New generation SAG”.

The exogenous gene of the present invention encodes a receptor that induces cell proliferation in response to ligand binding. Such endogenous genes are exemplified by those encoding receptors comprising a growth signal generator, such as cytokine receptors, including TPO receptor (c-Mpl) and G-CSF receptor (GCR). In the present invention, to regulate in vivo proliferation of the transplanted cells, it is preferred to use genes encoding artificial chimeric proteins that comprise a growth signal generator and a ligand-binding domain.

Cytokine receptors generate the growth signal through ligand-induced dimerization to induce cell proliferation of the cell. Therefore, it is preferred to use a chimeric protein that comprises (a) an extracellular domain of a receptor that dimerizes the chimeric protein in response to ligand binding, and (b) a growth signal generator that induces cell proliferation in response to the dimerization.

An “extracellular domain of a receptor that dimerizes the chimeric protein in response to ligand binding” can be exemplified by hormone-binding domains (e.g., estrogen or tamoxifen receptor) used in the previously reported SAG (Ito et al., Blood 1997, 90: 3884-92; Matsuda et al., Gene Ther 1999, 6: 1038-44; Xu et al., J Gene Med 1999, 1: 236-44; Nagashima et al., Biochem Biophys Res Commun 2003, 303: 170-6; Kume et al., J Gene Med 2003, 5: 175-81; Hanazono et al., Gene Ther 2002, 9: 1055-64), and the ligand-binding domain of EPOR of above-described SAG of the present invention. The ligand-binding domain, i.e., the extracellular domain of EPOR is particularly preferred for the method of the present invention.

The “growth signal generators” of the present invention are not restricted in any way so long as they induce cell proliferation of lymphohematopoietic cells in response to the binding of a ligand to the ligand-binding domain or the dimerization of the chimeric protein. Such growth signal generators include the cytoplasmic domain of a hematopoietic cytokine receptor, such as c-Mpl or GCR. However, the cytoplasmic domain of other cytokine receptors may also be used in the present invention, and those belonging to the cytokine receptor family encompassing GCR and c-Mpl as well as the tyrosine kinase receptor family (e.g., c-kit, flk2/flt3, etc.) can also be used.

The endogenous gene used for the present transplant can be constructed as described above under the item of “1. New generation SAG”. An SAG that encodes a fusion protein comprising the ligand-binding domain of EPOR and the cytoplasmic region of c-Mpl is a particularly preferred example of the exogenous gene used in the present invention.

The lymphohematopoietic cells of the present transplant preferably are transduced with a therapeutic gene. Suitable therapeutic genes are described above under the item of “2. Transplantation method”, and include genes that encode normal proteins ameliorating a symptom of a subject. For example, genes used in the gene therapy of chronic granulomatous disease, Gaucher's disease and Fanconi anemia are preferred.

The bone marrow transplant of the present invention comprises, in addition to the lymphohematopoietic cells, a pharmaceutically acceptable carrier. The pharmaceutically acceptable carrier is not restricted to any substance so long as it does not inhibit the transplantation of the cells, the expression of the receptor, ligand binding, proliferation of the cells and so on. Saline can be exemplified as a preferred pharmaceutically acceptable carrier.

Any other substance may be comprised in the bone marrow transplant of the present invention as needed, as long as it does not inhibit the transplantation of the cells, the expression of the receptor, ligand binding, proliferation of the cells and so on.

4. Kit

As described above, a gene encoding a receptor that induces cell proliferation in response to ligand binding find use in iBMT. Thus, the present invention provides a vector comprising such a gene as a kit for transplanting lymphohematopoietic cells into mammals. The kit of the present invention comprises: (a) a vector encoding a receptor that induces cell proliferation in response to ligand binding; and (b) a recording medium describing the use of the vector and lymphohematopoietic cells introduced with the vector for injection into the bone marrow cavity.

The present kit can be used for transplanting lymphohematopoietic cells, particularly pluripotent stem cells, for example, CD34⁺ cells selected from peripheral blood or bone marrow cells. The cells are preferably obtained from peripheral blood or bone marrow cells collected from the subject to be treated. The cells may be derived from any mammal; however, Primates are preferred. For example, Primates belonging to Prosimii and Anthropoidea, including human are particularly preferred.

The vector encoding a receptor of the present kit is suitably a viral vector. Any viral vector may be used; however, it preferably is a mammalian cell-infecting viral vector that is less toxic to host cells and achieves a high expression level of a transgene. Viral vectors, that can be used for the present invention include those recombinant viral vectors constructed by gene manipulation of adenovirus, adeno-associated virus, herpes simplex virus, retrovirus, lentivirus, Semliki forest virus, Sindvis virus, vaccinia virus, fowl pox virus and Sendai virus. The recombinant viral vectors may be generated by reconstituting virus particles through the expression of recombinant virus cDNA in host cells. Methods for preparing viral vectors are well known in the art and any method may be utilized for the present invention. See supra, under the item of “1. New generation SAG”.

The receptor encoded by the vector of the present invention induces cell proliferation in response to ligand binding. Exemplary receptors comprise a growth signal generator, such as cytokine receptors, including TPO receptor (c-Mpl) and G-CSF receptor (GCR). In the present invention, to regulate in vivo proliferation of the transplanted cells, artificial chimeric proteins that comprise a growth signal generator and a ligand-binding domain are preferred as the receptor to be encoded by the vector.

Cytokine receptors generate the growth signal through ligand-induced dimerization to induce cell proliferation of the cell. Therefore, it is preferred to use a chimeric protein that comprises (a) an extracellular domain of a receptor that dimerizes the chimeric protein in response to ligand binding, and (b) a growth signal generator that induces cell proliferation in response to the dimerization as the receptor.

An “extracellular domain of a receptor that dimerizes the chimeric protein in response to ligand binding” can be exemplified by hormone-binding domains (e.g., estrogen or tamoxifen receptor) used in the previously reported SAG (Ito et al., Blood 1997, 90: 3884-92; Matsuda et al., Gene Ther 1999, 6: 1038-44; Xu et al., J Gene Med 1999, 1: 236-44; Nagashima et al., Biochem Biophys Res Commun 2003, 303: 170-6; Kume et al., J Gene Med 2003, 5: 175-81; Hanazono et al., Gene Ther 2002, 9: 1055-64), and the ligand-binding domain of EPOR of above-described SAG of the present invention. The ligand-binding domain, i.e., the extracellular domain of EPOR is particularly preferred as the receptor of the present invention.

The “growth signal generators” of the present invention are not restricted in any way so long as they induce cell proliferation of lymphohematopoietic cells in response to the binding of a ligand to the ligand-binding domain or the dimerization of the chimeric protein. Such growth signal generators include the cytoplasmic domain of a hematopoietic cytokine receptor, such as c-Mpl or GCR. However, the cytoplasmic domain of other cytokine receptors may also be used in the present invention, and those belonging to the cytokine receptor family encompassing GCR and c-Mpl as well as the tyrosine kinase receptor family (e.g., c-kit, flk2/flt3, etc.) can also be used.

The vector of the present kit can be constructed as described above under the item of “1. New generation SAG”. A fusion protein encoded by an SAG that comprises the ligand-binding domain of EPOR and the cytoplasmic region of c-Mpl is a particularly preferred example of the receptor used in the present invention.

The kit of the present invention comprises, in addition to the lymphohematopoietic cells, a recording medium. The recording medium describes the use of the vector and lymphohematopoietic cells introduced with the vector for injection into the bone marrow cavity. It may be any recording medium including printable medium, such as paper and plastic; and computer readable medium, such as floppy disk (FD), compact disk (CD), digital versatile disc (DVD) and semiconductor memory. The description on the recording medium may be a full explanation how to use the vector and such, or just indication of a uniform resource locator (URL) of a file that publishes such explanation. The recording medium may be packaged together or separately with the cells, or when it is a printable medium, may be a package of the cells.

Any other material may be comprised in the kit of the present invention as needed. For example, the kit may further comprise a vector that encodes a therapeutic gene. Suitable therapeutic genes are described above under the item of “2. Transplantation method”, and include genes that encode normal proteins ameliorating a symptom of a subject. For example, genes used in the gene therapy of chronic granulomatous disease, Gaucher's disease and Fanconi anemia are preferred.

Furthermore, the kit may also comprise a ligand of the receptor encoded by the vector. For example, when the receptor comprises a ligand-binding domain of EPOR, EPO or EPO-mimetic peptide may be included in the kit.

Moreover, other agents, solutions, devices and such required for transplantation (e.g., syringe, needle, saline, wash solution, etc.), transduction of cells (e.g., container, culture media, etc.) and so on may be included in the kit of the present invention.

### BRIEF DESCRIPTION OF THE DRAWINGS

FIG. 1 depicts the structure of SAGs. GCRER: receptor encoded by the prototype SAG, i.e., a chimeric gene encoding the GCR as a growth-signal generator and the estrogen receptor hormone-binding domain (ER-HBD) as a molecular switch; and ΔY703F-GCRTmR: the G-CSF-binding domain is deleted from the GCR gene to abolish responsiveness to endogenous G-CSF, a point mutation (Y703F) is introduced in the GCR moiety to disrupt the differentiation signal generated by GCR, and another point mutation (G525R) is introduced in the ER-HBD moiety to evade responsiveness to endogenous estrogen without impairing responsiveness to a synthetic hormone tamoxifen. In the new SAGs, EPOR was utilized instead of the estrogen or tamoxifen receptor as a molecular switch. The intracellular domain of wild-type EPOR (EPORwt) gene was replaced by that of the GCR or thrombopoietin receptor (c-Mpl) gene as a growth-signal generator.

FIG. 2 depicts graphs showing Ba/F3 cell growth efficiently stimulated by EPO-driven SAG. FIG. 2(A) shows the EPO-dependent growth of Ba/F3 cells by the introduction of the EPO-driven SAG. Ba/F3 cells were transduced with EPORwt (closed triangle), EPORGCR (closed square) or EPORMpl gene (closed circle) each along with the EYFP gene by bicistronic retroviral vectors. YFP-positive cells were sorted (>98%) and treated with EPO at various concentrations. The proliferation assay (see Materials and Methods of Example 1) was performed on day 0 and day 2, and the ratio of day 2 A₄₉₀-A₆₅₀ to day 0 A₄₉₀-A₆₅₀ (means±SD of triplicate) is shown. The arrow indicates the physiological range of EPO concentrations in human plasma. FIG. 2(B) shows that the EPO-driven SAG triggers higher levels of cell proliferation than the steroid-driven SAG. The parental Ba/F3 cells (open diamond) were cultured in the presence of IL-3 (10 ng/ml). Ba/F3 cells transduced with EPORwt (closed triangle), EPORGCR (closed square) or EPORMpl gene (closed circle) were cultured in the presence of EPO (10 ng/ml). Ba/F3 cells transduced with the ΔGCRTmR gene (open triangle) were cultured in the presence of tamoxifen (10⁻⁷ M). Accumulative cell numbers calculated with means of triplicate are shown in log scale.

FIG. 3 depicts a graph showing that EPORMpl is the most potent amplifier for human cord blood CD34⁺ cells. Human cord blood CD34⁺ cells were transduced with EPORwt (closed triangle), EPORGCR (closed square) or EPORMpl gene (closed circle) each along with the EYFP gene by bicistronic retroviral vectors. Untransduced cells are also shown (open diamond). The cells were then cultured in IMDM supplemented with 10% FBS and 10 ng/ml EPO. Virtually all the cells (>95%) became YFP-positive by week 2. Accumulative cell numbers calculated with the means of triplicate are shown in log scale.

FIG. 4 depicts the result of flow cytometry showing the most efficient ability of EPORMpl to preserve c-Kit⁺ cells. Human cord blood CD34⁺ cells were transduced with EPORwt (black), EPORGCR (gray) or EPORMpl gene (white) by the same retroviral vectors in FIG. 3. The cells were then cultured in IMDM supplemented with 10% FBS and 10 ng/ml EPO. On the indicated days, aliquots of the cells were examined for c-Kit expression by flow cytometry. The percentages of cKit-positive cells are shown.

FIG. 5 depicts graphs showing that the EPORMpl expands clonogenic progenitor cells most efficiently. Human cord blood CD34⁺ cells were transduced with EPORwt, EPORGCR or EPORMpl gene by the same retroviral vectors in FIG. 3. The untransduced and transduced cells were then cultured in IMDM supplemented with 10% FBS and 10 ng/ml EPO for 7 days. The cells before (day 0) and after (day 7) the liquid culture were plated in methylcellulose medium in the presence of EPO alone and the resultant colonies were counted. FIG. 5(A) shows the total myeloid clonogenic progenitor cell (CFU) numbers per culture. FIG. 5(B) shows the total erythroid CFU numbers per culture.

FIG. 6 depicts the result of flow cytometry showing that the CD34⁺ cells expanded by EPORMpl show the most balanced expression of multilineage surface markers. Human cord blood CD34+cells were transduced with EPORwt, EPORGCR or EPORMpl gene by the same retroviral vectors in FIG. 3. After 14-day liquid culture with 10% FBS and 10 ng/ml EPO, the transduced cells were examined for the expression of glycophorin A (erythroid marker), CD15 (myeloid marker) and CD41 (megakaryocyte marker) by flow cytometry. The percentages of marker-positive cells are shown.

FIG. 7 depicts graphs showing that the gene-modified hematopoietic cells can be expanded by treatment with EPO in vivo in mice. Murine bone marrow cells were harvested from 5-fluorouracil-treated mice and transduced with the retroviral vector expressing both EPORMpl and YFP, or YFP alone as a control. The transduced cells were transplanted into irradiated mice. The percentages of YFP-positive cells in the peripheral blood are shown in the EPORMpl group (FIG. 7A) or the YFP control group (FIG. 7B). In each group, mice were divided into two subgroups: EPO-treated subgroup (n=6, 200 IU/kg, three times a week, close bars) and EPO-untreated subgroup (n=4 or 6, open bars). The gray arrows in FIGS. 7A and 7B indicate the week of EPO administration. The increase in YFP-positive cell-s in the EPO-treated mice was significant at week 10 (4 weeks after the initiation of EPO administration)(*, p<0.05).

FIG. 8 depicts a photograph (A) and schematic diagram (B) of the iBMT method. Needles were inserted at both ends of the limb bones (femurs and humeri) and the bone marrow cavity was gently irrigated with saline without inflicting extra-pressure. Gene-modified CD34⁺ cells were then directly injected into the bone marrow through the needle on one side.

FIG. 9 depicts graphs showing the in vivo marking after iBMT and intravenous transplantation, without marrow conditioning. CD34⁺ cells were transduced with non-expression retroviral vector PLI and returned by iBMT (A, IB3048 and B, IB3053) or by intravenous transplantation (C, V0065 and D, V1007) without conditioning. The upper row shows ratios for provirus-positive CFUs to β-actin-positive CFUs taken from the non-transplanted marrow at time points indicated by arrows. Overall number of provirus-positive CFUs versus overall number of β-actin-positive CFUs was 74/522 (14.2%) for iBMT (A and B) and 15/274 (5.5%) for the intravenous transplantation (C and D). The lower diagram shows percentages of gene-modified cells in the peripheral blood as assessed by quantitative PCR.

FIG. 10 depicts graphs showing the expansion of SAG-transduced cells upon treatment with EPO after iBMT. CD34⁺ cells transduced with SAG were returned to each animal by iBMT without conditioning. The animal S9042 (A) and S3047 (B) received EPO at 200 IU/kg once or twice daily (indicated by closed bars). The upper row shows ration of provirus-positive CFUs to β-actin-positive CFUs taken from the non-transplanted marrow at time points indicated by arrows. The lower, diagram shows percentage of gene-modified cells in the peripheral blood as assessed by quantitative PCR.

FIG. 11 depicts photographs showing high-level, multilineage and polyclonal expansion of gene-modified cells in the peripheral blood after iBMT with SAG in non-conditioned recipients. FIG. 11A shows the photograph of in situ PCR for the provirus. Peripheral blood nucleated cells were collected from animal S9042 that received EPO at day 89 post-transplantation. Many SAG-transduced cells (stained in black) were detected by in situ PCR. FIG. 11B shows the result of lineage analysis by semi-quantitative PCR. DNA from granulocytes (Gr), and T- and B-lymphocytes sorted from animal S9042 that received EPO at day 91 post-transplantation was examined for the provirus by semi-quantitative PCR. Positive controls were included corresponding to 0.2, 0.6, 2.0, 6.0 and 20% of transduced cells in peripheral blood. FIG. 11C shows the result of clonal analysis by LAM-PCR. Genomic DNA from peripheral blood of animals that received EPO(S9042 at day 90 and S3047 at day 150 post-transplantation) was analyzed by LAM-PCR. Each band indicates different integrants. Negative control was genomic DNA from a naive monkey. M: molecular weight marker.

FIG. 12 depicts the result of dual genetic marking study. CD34⁺ cells from monkey D8058 were split into two equal aliquots; one aliquot was transduced with SAG vector (indicated by open circles) and the other with non-expression PLI vector (indicated by closed circles). Both aliquots were returned together to the bone marrow cavity by iBMT without conditioning. EPO (200 IU/kg; twice daily) was administered from the day after transplantation (indicated by a closed bar).

FIG. 13 depicts a graph showing positive blastogenic response of lymphocytes to SAG. Peripheral blood mononuclear cells (responder cells) were isolated from monkey D8058 at day 169 post-transplantation (FIG. 12) and cocultured with stimulator cells. The stimulator cells were autologous stromal cells untransduced or retrovirally transduced with PLI, SAG or human EPO receptor cDNA followed by irradiation with 4,000 cGy. After 5 days of culture, the blastogenesis of responder cells was assessed by counting the [³H]thymidine incorporation into responder cells. The average±SD of triplicate experiments is shown. N.S.: not significant.

### BEST MODE FOR CARRYING OUT THE INVENTION

The following examples are presented to illustrate the present invention and to assist one of ordinary skill in making and using the same. The examples are not intended in any way to otherwise limit the scope of the invention.

Although methods and materials similar or equivalent to those described herein can be used in the practice or testing of the present invention, suitable methods and materials are described below. Any patents, patent applications and publications cited herein are incorporated by reference.

### Example 1

Material and Methods

(1) Cell Lines

Ba/F cells were maintained in Dulbecco's modified Eagle's medium (DMEM; Gibco-BRL, Grand Island, N.Y.) supplemented with 10% fetal bovine serum (FBS; Gibco-BRL), 1% penicillin/streptomycin (Gibco-BRL) and 1 ng/ml recombinant mouse IL-3 (rmIL-3; Gibco-BRL). The ecotropic packaging cell line BOSC23 (Pear et al., Proc Natl Acad Sci USA 1993, 90: 8392-6) and human embryonic kidney 293T cells were maintained in DMEM containing 10% FBS (Gibco-BRL) and 1% penicillin/streptomycin (Gibco-BRL).

(2) Plasmid Construction

The wild-type human erythropoietin receptor (EPORwt) cDNA was obtained from pCEP4-EPOR (kindly provided by Dr. R. Kralovics, University of Alabama, UK) (Kralovics et al., J Clin Invest 1998, 102: 124-9). The fragment containing the murine phosphoglycerate kinase (pgk) promoter and neomycin phosphotransferase gene (neo) (EcoRI-BamHI) in the retroviral plasmid pMSAV2.2 (kindly provided by Dr. R. G. Hawley, University of Toronto, Canada) (Hawley et al., Gene Ther 1994, 1: 136-8) was replaced by the EPORwt cDNA (EcoRI-BamHI) to construct pMSCV-EPORwt.

pMSCV-EPORGCR and pMSCV-EPORMpl were constructed as follows. The cytoplasmic region of murine GCR cDNA was obtained by PCR using pMSCV-ΔY703FGCRER as a template (Matsuda et al., Gene Ther 1999, 6: 1038-44) with the primer pair 5′-AAG GAT CCA AAC GCA GAG GAA AGA AGA CT-3′ and 5′-AAG TCG ACC TAG AAA CCC CCT TGT TC-3′. The cDNA encoding the cytoplasmic region of human TPO receptor (c-Mpl) was obtained by PCR using pcDNA3.1-c-Mpl (provided by Dr. M. Takatoku, Jichi Medical School, Tochigi, Japan) (Takatoku et al., J Biol Chem 1997, 272: 7259-63) as a template with the primer pair 5′-AAG GAT CCA GGT GGC AGT TTC CTG CA-3′ and 5′-CGG TCG ACT CAA GGC TGC TGC CAA TA-3′. The fragment containing the extracellular and transmembrane regions of human EPOR cDNA was obtained by PCR using pCEP4-EPOR as a template with the primer pair 5′-CTC GGC CGG CAA CGG CGC AGG GA-3′ and 5′-AAG GAT CCC AGC AGC GCG AGC ACG GT-3′. The fragment containing the extracellular and transmembrane regions of human EPOR cDNA and the fragment containing the cytoplasmic region of murine GCR or human c-Mpl were cloned into the EcoRI-SalI site of pBluescript SK (pSK; Stratagene, La Jolla, Calif.) to construct pSK-EPOGCR or pSK-EPOMpl, respectively. The pgk promoter/neo cassette (EcoRI-SalI) in pMSCV was replaced by the EcoRI-SalI fragment containing the EPORGCR or EPORMpl cDNA from pSK-EPORGCR or pSK-EPORMpl. The resultant constructs were designated pMSCV-EPORGCR and pMSCV-EPORMpl, respectively.

pMSCV-EPORwt-ires-mitoEYFP, pMSCV-EPORGCR-ires-mitoEYFP and pMSCV-EPORMpl-ires-mitoEYFP were constructed as follows. The IRES sequence derived from pIRES-EGFP (Clontech, Palo Alto, Calif.) and mitoEYFP cDNA derived from pEYFP-Mito (Clontech) were inserted into the PstI-BamHI site and SpeI-NotI site of pSK, respectively. Thus, pSK-ires-mitoEYFP was obtained. The mitoEYFP cDNA encodes the enhanced yellow fluorescent protein (enhanced YFP, EYFP) linked to a mitochondria localization signal sequence so that EYFP is sequestered inside the mitochondria, thus circumventing the presumed toxicity of YFP (Huang et al., FEBS Lett 2000, 487: 248-51). The blunted fragment encoding the ires-mitoEYFP cDNA was ligated into the ClaI blunted site of pMSCV-EPORwt, pMSCVGCR and pMSCV-EPORMpl to obtain pMSCV-EPORwt-ires-mitoEYFP, pMSCV-EPORGCR-ires-mitoEYFP and pMSCV-EPORMpl-ires-mitoEYFP, respectively. Finally, the sequences of the constructed plasmids were certified by sequence analysis.

(3) Retroviral Vectors

To obtain ecotropic retroviral vectors, BOSC23 cells were transfected with mouse stem cell virus (MSCV)-based retroviral plasmids (derivatives of pMSCV, see above) using Lipofectamine Plus (Invitrogen, San Diego, Calif.) according to the manufacturer's protocol and the supernatants containing the ecotropic retroviral vectors were harvested 48 to 72 hr after transfection. The titer was assessed 1×10⁶/ml by RNA dot-blot. To obtain amphotropic retroviral vectors, 293T cells were transfected with MSCV-based retroviral plasmids along with pCL-Ampho (Imugenex, San Diego, Calif.) using Lipofectamine Plus (Invitrogen) and the supernatants containing the amphotropic retroviral vectors were harvested 48 to 72 hr after transfection. The titer was assessed 1×10⁶/ml by RNA dot-blot.

(4) Retroviral Transduction and Culture

BA/F3 cells were suspended in 1 ml retroviral supernatant containing 10 ng/ml rmIL-3 at a density of 1×10⁵ cells/ml, and transferred to 12-well plates coated with 20 μg/cm² of RetroNectin (Takara Bio, Shiga, Japan) (Hanenberg et al., Nat Med 1996, 2: 876-82). The cells were incubated at 37° C. in a humidified atmosphere of 5% CO₂ for 24 hr. During this period, culture medium was replaced by fresh viral supernatant twice (every 12 hr). After retroviral infection, YFP-positive cells were isolated using EPICS ELITE cell sorter (Coulter, Miami, Fla.) according to the manufacturer's instructions. The purity of sorted EFP-positive cells was greater than 98%. The sorted Ba/F3 cells were subjected to further liquid culture (described above) or cell proliferation assays (see below).

Human cord blood CD34⁺ cells (BioWhittaker, Walkersville, Md.) were thawed and placed in 12-well plates coated with 20 μg/cm² of RetroNectin (Takara Bio) and cultured for 24 hr at 37° C. with 5% CO in Iscove's modified Dulbecco's medium (IMDM; Gibco-BRL) supplemented with 10% FBS (Hyclone, Logan, Utah), 50 ng/ml recombinant human interleukin 6 (rhIL-6; Ajinomoto, Osaka, Japan), 100 ng/ml recombinant human stem cell factor (Research Diagnostic, Flanders, N.J.) and 100 ng/ml recombinant thrombopoietin (rhTPO; Kirin, Tokyo, Japan). The cells were then resuspended in 1 ml viral supernatant containing the same cytokines as described above at a starting density of 1×10⁵ cells/ml. During the transduction period (48 hr), culture medium was replaced by fresh viral supernatant 4 times (every 12 hr). After retroviral transduction, human cord blood CD34⁺ cells were washed twice and cultured in IMDM medium containing 10% FBS (Hycone) and 1% penicillin/streptomycin in the presence of 10 ng/ml EPO in a 37° C. 5% CO₂ incubator. The cells were subjected to flow cytometry or colony assay (see below) on the indicated days.

(5) Cell Proliferation Assay

Ba/F3 proliferation assay was performed using CellTier 96 Aqueous One Solution Cell Proliferation Assay (Promega, Madison, Wis.) according to the manufacturer's instructions. Specifically, 20 μl MTS

(3[4,5-dimethylthiazol-2-yl]-5-[3-carboxymethoxyphenyl]-2-[4-sulfophenyl]-2H-tetrazolium)-labeling mixture was added to each well of 96-well dishes containing cells to be assayed. Following incubation at 37° C. for 2 hr, spectrophotometric absorbance was measured at a wavelength of 490 nm and 650 nm. A₄₉₀-A₆₅₀ values were used to determine Ba/F3 cell proliferation. Experiments were conducted in triplicate.

(6) Flow Cytometry

Human cord blood CD34⁺ cells were washed and resuspended in CellWASH (Becton Dickinson, San Jose, Calif.). The cells were then incubated with phycoerythrin (PE)-labeled anti-c-Kit (Nichirei, Tokyo, Japan), PE-labeled anti-glycophorin A (Nichirei), PE-labeled anti-CD41 (Nichirei) or PE-labeled anti-CD15 (Immunotech, Marseille, France) at 4° C. for 30 min. The cells were washed once and subjected to FACSCalibur (Becton Dickinson) using excitation at 488 nm. Untransduced cells served as negative control.

Mouse blood cells were suspended in ACK lysis buffer (155 mM NH₄Cl, 10 mM KHCO₃ and 0.1 mM EDTA; Wako, Osaka, Japan) to dissolve red blood cells as mouse blood samples. The cells were washed once and subjected to FACSCalibur (Becton Dickinson) using excitation at 488 nm.

(7) Colony Assay and PCR

Human cord blood CD34⁺ cells were plated in 35-mm dishes with α-minimum essential medium (Gibco-BRL) containing 1.2% methylcellulose (Shin-Etsu Chemicals, Tokyo, Japan) supplemented with 20% FBS (Intergen, Purchase, N.Y.) and 1% bovine serum albumin (Sigma, St. Louis, Mo.) in the presence of 100 ng/ml rh stem cell factor (SCF), 100 ng/ml rhIL-6 and 100 ng/ml recombinant human interleukin 3 (rhIL-3; PeproTech, London, UK), or in the presence of 20 ng/ml of recombinant human erythropoietin (rhEPO) alone. After incubation for 14 days at 37° C. in a humidified atmosphere of 5% CO₂, colonies were scored under an inverted microscope. The experiments were performed in triplicate.

Colonies in methylcellulose culture were picked up under an inverted microscope, suspended in 50 μl of distilled water, and digested with 20 μg/ml protenase K (Takara Bio) at 55° C. for 1 hr followed by incubation at 99° C. for 10 min. PCR was performed to amplify the 351-bp sequence using the EYFP sense primer (5′-CGT CCA GGA GCG CAC CAT CTT C-3′) and antisense primer (5′-AGT CCG CCC TGA GCA AAG ACC-3′). To certify the initial DNA amounts, the β-actin genomic DNA fragment was simultaneously amplified using the sense primer (5′-CAT TGT CAT GGA CTC TGG CGA CGG-3′) and antisense primer (5′-CAT CTC CTG CTC GAA GTC TAG GGC-3′). Amplification conditions were 95° C. for 1 min, 55° C. for 30 sec and 72° C. for 30 sec with 35 cycles.

(8) Mouse Transplantation

Eight-week old C57B1/6 mice (Charles River Japan, Yokohama, Japan) intraperitoneally received 150 μg/kg 5-fluorouracil (Sigma). Forty-eight hr after injection, bone marrow cells were harvested from the femora of each mouse. Cells were cultured in IMDM (Gibco-BRL) containing 20% FBS (Hyclone) and 20 ng/ml rhIL-6 and 100 ng/ml recombinant rat SCF (provided by Amgen, Thousand Oaks, Calif.) for 48 hr. The cells were then placed in 6-well plates coated with 20 μg/cm² of RetroNectin (Takara Bio) and resuspended in IMDM (Gibco-BRL) supplemented with 10% FBS (Hyclone) and the aforementioned cytokines at a starting density of 5×10⁵ cells/ml. During the transduction period (48 hr), culture medium was replaced by fresh viral supernatant 4 times (every 12 hr). The cells were harvested after a total of 96 hr (4 days) in culture, washed 3 times with phosphate-buffered saline (PBS), and injected into 8-week-old female C57/B16 mice that had been irradiated with 800 cGy. After transplantation, some mice received recombinant mouse EPO (rmEPO; 200 IU/kg; Roche Diagnostics) at a total volume of 100 μl via the tail vein three times a week. To avoid development of anemia after drawing blood from the transplanted mice, blood was transfused into the mice via the tail vein at the time of blood drawing. The blood for transfusion was drawn from donor-C57/B16 mice and pooled. It was irradiated at 20 Gy and diluted with physiological salt solution prior to transfusion. Peripheral blood mononuclear cells of the recipient mice were analyzed for EYFP expression by flow cytometry.

Results

(1) New Generation SAG

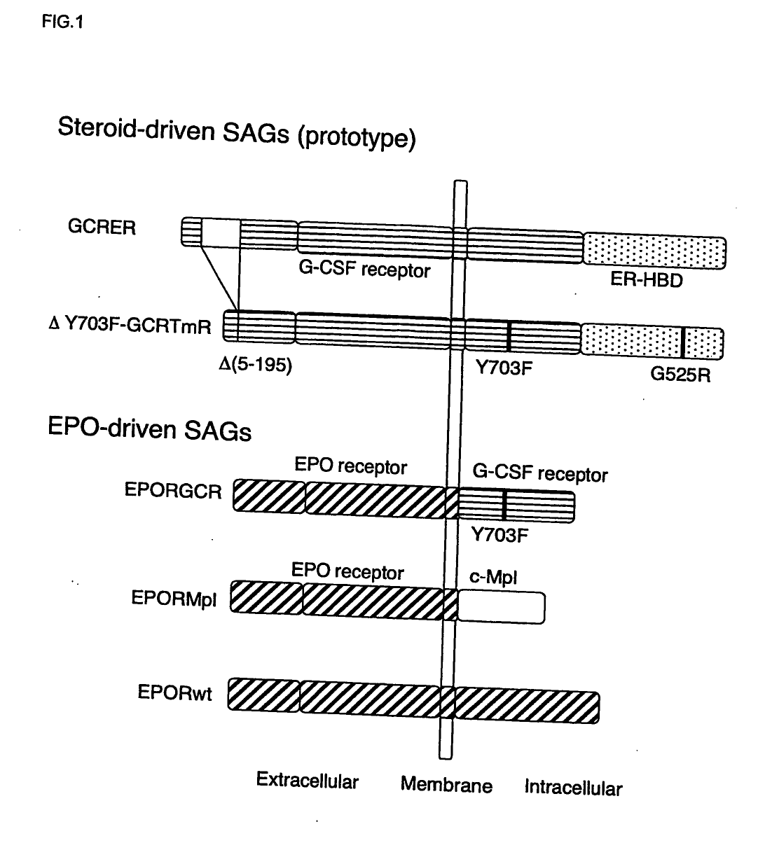

The structures of SAGs are shown in FIG. 1. One of the prototype SAG (steroid-driven SAGs) is encoded by a chimeric gene that encodes GCR and the estrogen receptor hormone-binding domain. In GCR, the ligand (G-CSF)-binding domain was deleted to remove the responsiveness to endogenous G-CSF (Ito et al., Blood 1997, 90: 3884-92). The tyrosine residue at the 703rd amino acid in GCR was replaced by phenylalanine to hamper the differentiation signal (Matsuda et al., Gene Ther 1999, 6: 1038-44). In addition, another mutation (G525R) was introduced in the estrogen receptor hormone-binding domain to evade the responsiveness to endogenous estrogen without impairing the responsiveness to synthetic hormones such as tamoxifen (Xu et al., J Gene Med 1999, 1: 236-44). In this study, the inventors constructed new generation SAGs wherein EPOR is utilized instead of the estrogen or tamoxifen receptor as the molecular switch. Two types of EPO-driven SAG, EPORGCR and EPORMpl, encoded by chimeric genes that contain the GCR and the TPO receptor (c-Mpl) genes, respectively, as the growth-signal generator were constructed.

(2) In Vitro Effects of EPO-Driven SAG on Ba/F3

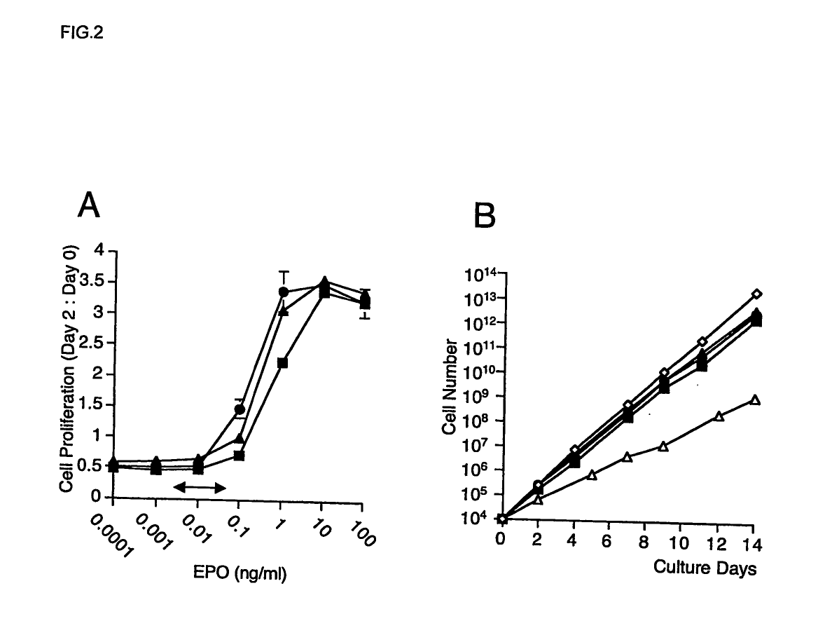

Bicistronic retroviral vectors that express the EPO-driven SAG or wild-type EPOR (EPORwt) gene as the first cistron and the EYFP gene as the second cistron were constructed. The vectors were infected into Ba/F3 cells. Ba/F3 cell is a mouse pro-B cell line and requires IL-3 for growth. YFP-positive cells were isolated (>98% purity) and stimulated with EPO at various concentrations (FIG. 2A). All the cells acquired the ability of EPO-dependent growth and were able to proliferate even in the absence of IL-3. Ba/F3 cells expressing either EPORwt, EPORGCR or EPORMpl reached the maximum growth levels by adding 1 to 100 ng/ml EPO (FIG. 2A). Endogenous EPO will not induce a significant proliferative response on the cells, since the physiological range of serum EPO concentration is below 0.1 ng/ml.

The EPO- and steroid-driven SAGs were compared in terms of their ability to expand Ba/F3 cells. The Ba/F3 cells expressing the EPO-driven SAGs were cultured in the presence of 10 ng/ml EPO and those expressing the steroid-driven SAG were cultured in the presence of 10⁻⁷ M tamoxifen (Xu et al., J, Gene Med 1999, 1: 236-44). The Ba/F3 cells expressing either of the two EPO-driven SAGs proliferated in the presence of EPO to the same extent as the parental Ba/F3 cells in the presence of IL-3. Of note, EPOGCR expanded Ba/F3 cells by around 10⁴-fold more than the steroid-driven counterpart (ΔGCRTmR) after 2 weeks of culture (FIG. 2B), indicating that the molecular switch using EPOR is more efficient than that using the tamoxifen receptor despite the inclusion of the same signal generator (GCR) in the SAGs. Thus, EPO-driven SAGs were used for subsequent experiments.

(3) In Vitro Effect of the EPO-Driven SAGs on Human CD34⁺ Cells

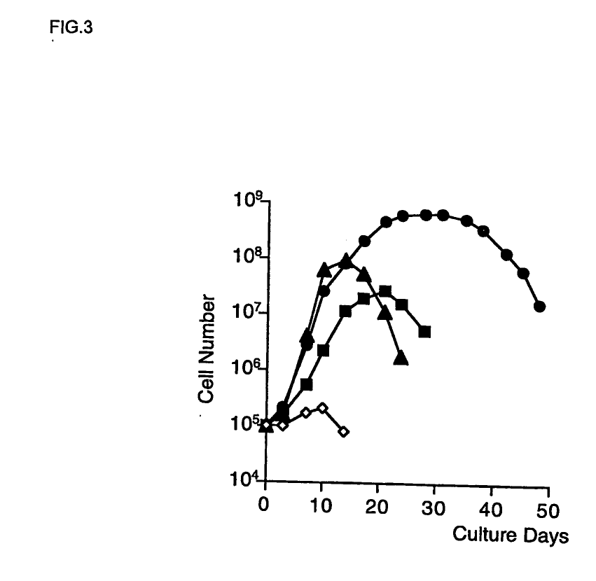

To examine which of GCR or c-Mpl is the more suitable signal generator for EPO-driven SAG, human cord blood CD34⁺ cells were used as targets. CD34⁺ cells were transduced with bicistronic retroviral vectors that express EPO-driven SAG as the first cistron and the EYFP gene as the second cistron. After transduction, 27.3±4.7% of the cells fluoresced (YFP-positive). The transduced CD34⁺ cells were then cultured in liquid medium in the presence of EPO. The fraction of YFP-positive cells increased over time, and virtually all (>95%) of the cells became YFP-positive during a 2-week culture with EPO. This suggests that the EPO-driven SAGs are able to confer a growth advantage on human CD34⁺ cells. As shown in FIG. 3, although the cells transduced with EPORwt proliferated most quickly, the cell number already began to decrease within 2 weeks after culture initiation. The cells transduced with EPORGCR grew slowly compared with the others. However, began to decrease in number by week 3. On the other hand, the cells transduced with EPORMpl proliferated the longest (1 month) in the presence of EPO and the cell number increased by 10⁴-fold over this period.

(4) Characterization of c-Mpl Signal of SAG

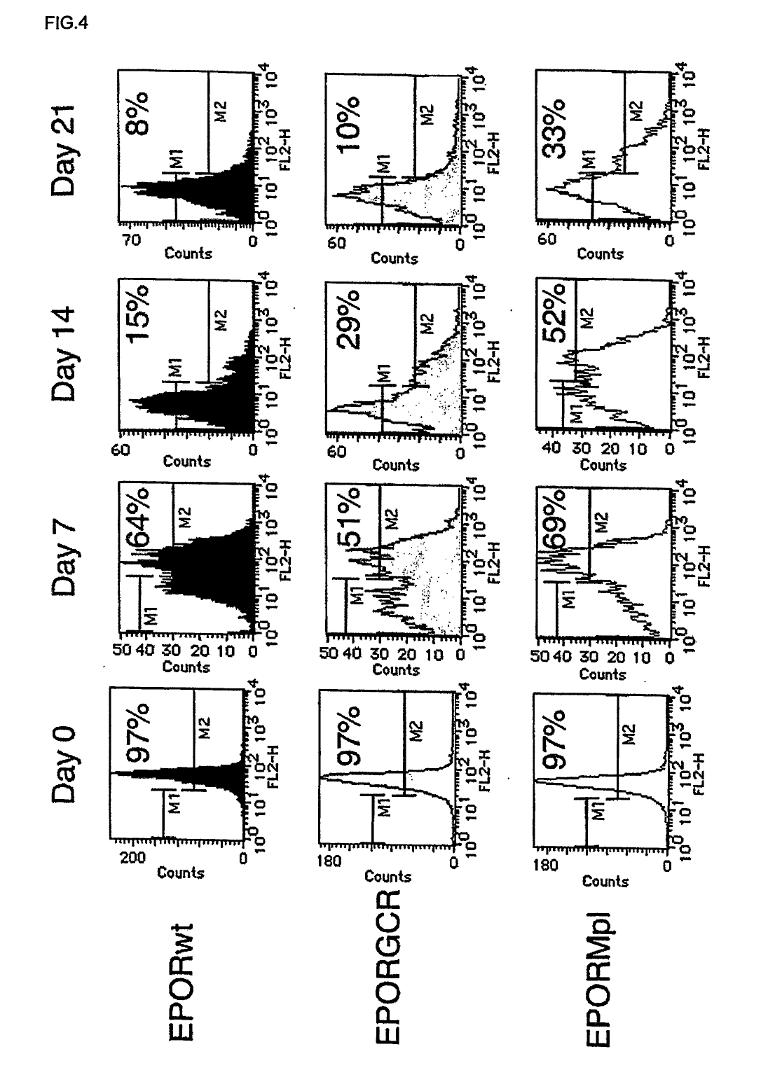

The transduced CD34⁺ cells were then examined for their expression of c-Kit, a primitive hematopoietic cell marker, by flow cytometry (FIG. 4). The c-Kit⁺ fraction decreased over time, implying that the cells differentiated during culture. The c-Kit⁺ fraction in the cells transduced with EPORMpl, however, was relatively high (33%) at week 3 in liquid culture, whereas that in the cells transduced with EPORwt or EPORGCR decreased to 10% or lower at the same time point. These results demonstrate that the c-Mpl signal preserved more c-Kit⁺ immature hematopoietic cells than the other signals.

To examine the EPO-driven SAGs for their ability to expand hematopoietic progenitor cells, CD34⁺ cells transduced with the SPO-driven SAGS were cultured in semisolid (methylcellulose) media in the presence of multiple cytokines (IL-3, IL-6 and SCF) or EPO alone. The results are summarized in Table 1.

TABLE 1
Colony formation by human cord blood CD34⁺ cells transduced
with EPO-driven SAGs

| | IL-3 (100 ng/ml) | | | |
|---|---|---|---|---|
| | IL-6 (100 ng/ml) | | | |
| | SCF (100 ng/ml) | | EPO (20 ng/ml) | |
| | | Provirus- | | Provirus- |
| | Number of | positive | Number of | positive |
| Transgene | colonies* | colonies** | colonies* | colonies** |
| EPORwt-YFP | 62 ± 11 | 5/16 | 15 ± 3 | 15/16 |
| | | (31%) | | (94%) |
| EPORGCR-YFP | 54 ± 8 | 6/16 | 24 ± 1 | 16/16 |
| | | (38%) | | (100%) |
| EPORMpl-YFP | 54 ± 9 | 4/16 | 31 ± 6 | 15/16 |
| | | (25%) | | (94%) |
| YFP | 49 ± 4 | 8/16 | 12 ± 1 | 9/16 |
| | | (50%) | | (56%) |
| untransduced | 53 ± 4 | ND | 17 ± 1 | ND |

*Colony number out of 200 cells is shown. Each value represents mean ±SD of triplicate culture.
**Individual colony DNA was subjected to PCR for the proviral YFP and genomic β-actin sequences and the ratio of the provirus-positive colony number to the β-actin-positive colony number is shown.

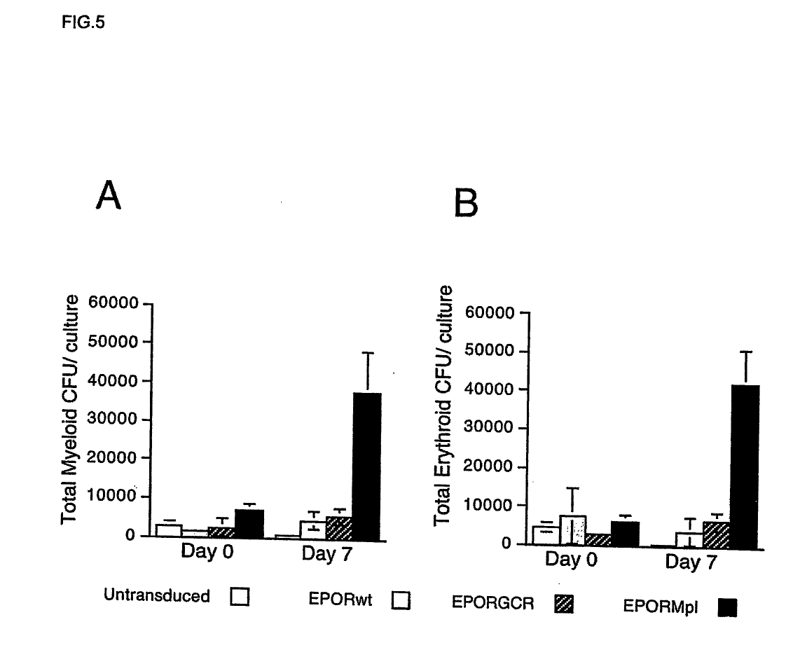

The cells transduced with the EPO-driven SAGs formed many colonies in the presence of EPO and almost all of them (94 to 100%) contained the provirus as assessed by individual colony PCR. In contrast, 25 to 38% of the colonies formed by cells in the presence of multiple cytokines contained the provirus. This result shows that the EPO-driven SAGs are able to confer an EPO-dependent growth advantage at the level of clonogenic progenitor cells. The cells transduced with the EPO-driven SAGs before (day 0) and after (day 7) liquid culture with EPO were placed in semisolid media in the presence of EPO without other cytokines, and the resultant myeloid and erythroid colonies were counted. As shown in FIG. 5, during the liquid culture with EPO, the transduction by EPORMpl resulted in the higher levels of clonogenic progenitor cell expansion by more than 10-fold.

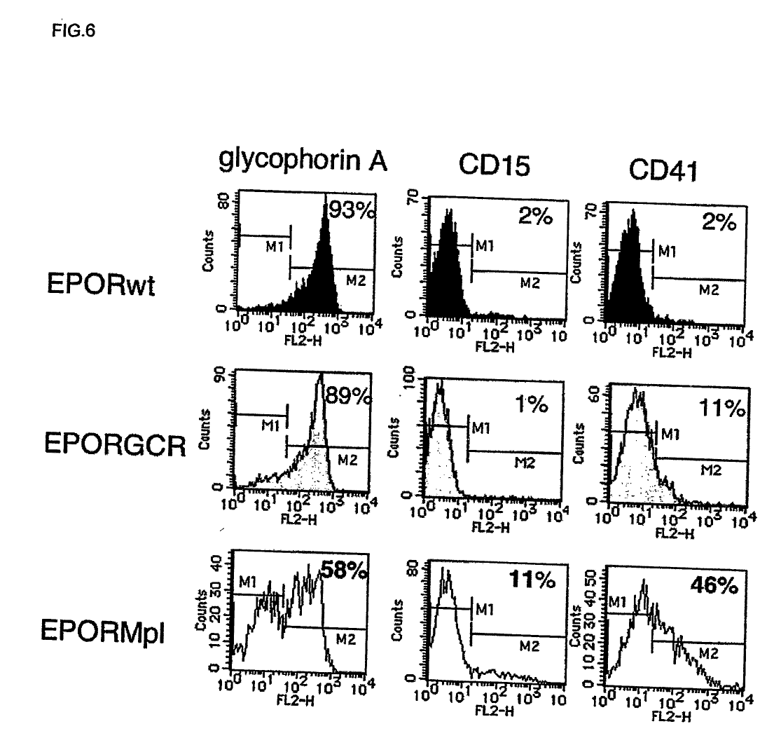

Next, cells transduced with the EPO-driven SAGs were examined for their specific lineage preference after liquid culture with EPO. The transduced CD34⁺ cells were cultured in liquid medium containing EPO. During the culture, the expression of various differentiation markers was examined by flow cytometry (FIG. 6). As expected, the erythroid marker (glycophorin A) was expressed in almost all (93%) cells transduced with EPORwt at day 14. The myeloid marker (—CD15) was expressed in 24% of cells transduced with EPORGCR at day 7 (data not shown), however, fell to 1% by day 14. Thus, EPORGCR induced very few cells to differentiate toward the myeloid lineage despite the inclusion of the GCR moiety as the signal generator. One reason may be that a point mutation (Y703F) was introduced into the GCR cDNA to attenuate the granulocytic differentiation signal (FIG. 1) (Matsuda et al., Gene Ther 1999, 6: 1038-44). On the other hand, cells transduced with EPORMpl expressed all of these markers at relatively high levels at day 14; the megakaryocytic marker (CD41) (46%), glycophorin A (58%) and CD15 (11%). Thus, the cells expanded by the c-Mpl signal showed the most balanced expression of myeloid, erythroid and megakaryocyte markers. Therefore, EPORMpl was decided as the SAG for subsequent in vivo experiments in mice.

(5) In Vivo Expansion of Gene-Modified Cells

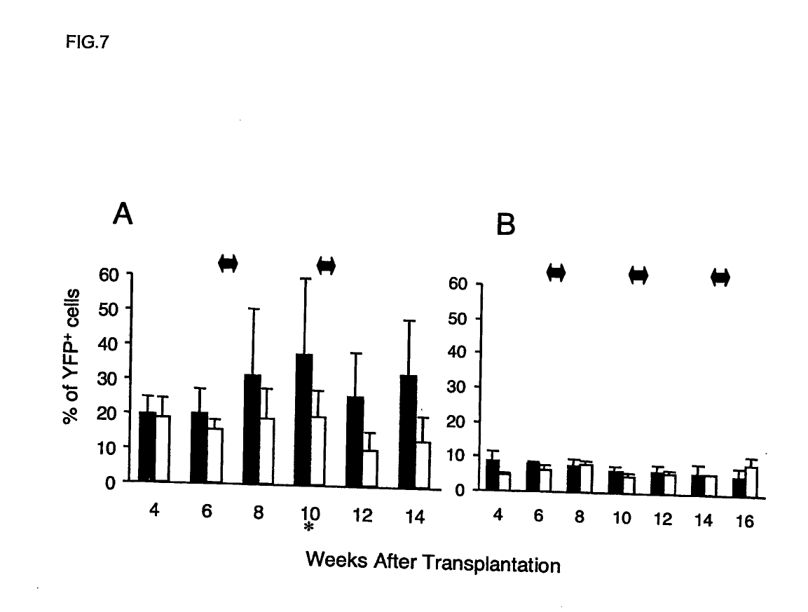

Finally, the in vivo efficacy of the EPORMpl-type SAG was examined in mice. Murine bone marrow cells were harvested from 5-fluorouracil-treated mice and transduced with the MSCV-based vector expressing both EPORMpl and YFP, or YFP alone as a control. The transduced cells were transplanted into irradiated mice, and after hematopoietic reconstitution, YFP expression was examined in the peripheral blood by flow cytometry to see whether the EPORMpl-transduced cells increase in response to EPO administration. In mice, however, even drawing a small volume of blood will result in the elevation of endogenous EPO concentrations (Oishi et al., J Vet Med Sci 1993, 55: 51-8; Chapel et al., Exp Hematol 2001, 29: 425-31). Sequential blood-drawing was also confirmed to cause an elevation of endogenous serum EPO concentrations in mice (data not shown). Therefore, drawing blood from the transplanted mice may result in the expansion of transduced hematopoietic cells. To avoid development of anemia due to blood drawing, the mice were transfused at the time of blood drawing. As a result, the mice did not develop anemia, and thus the elevation of endogenous EPO concentration was prevented. In the group receiving EPORMpl, YFP-positive cells increased in response to EPO administration (n=6), although YFP-positive cells remained unchanged without EPO administration (n=4) (FIG. 7A). On the other hand, in the control group (n=6) receiving YFP alone without EPORMpl, YFP-positive cells remained unchanged at around 10% in the peripheral blood regardless of EPO administration (FIG. 7B). In the mice receiving EPORMpl, a significant increase (paired t-test, p<0.05) in YFP-positive cells was observed 4 weeks after the initiation of EPO administration (FIG. 7A). The increase was attributable to that in granulocytes and monocytes (data not shown). However, the increase seemed transient, as a significant increase was no longer observed at further time points.

Thus, EPORMpl was demonstrated to confer an EPO-dependent growth advantage on the transduced hematopoietic cells in vivo in a mouse transplantation model. It should be noted that EPORMpl contains the human c-Mpl and may not have worked well in mouse cells. It would be more predictive to examine the efficacy of the EPORMpl in nonhuman primates. In mice, the increase of transduced cells with EPORMpl seemed transient, as was reported for chimeric genes by other investigators (Jin et al., Nat Genet 2000, 26: 64-6; Neff et al., Blood 2002, 100: 2026-31).

### Example 2

Material and Methods

(1) Animals

Cynomolgus monkeys (Macaca fascicularis) were housed and handled in accordance with the rules for animal care and management of the Tsukuba Primate Center and the guiding principles for animal experiments using nonhuman primates formulated by the Primate Society of Japan. The animals (2.5 to 5.6 kg, 3 to 5 years) were certified free of intestinal parasites and seronegative for simian type-D retrovirus, herpes virus B, varicella-zoster-like virus and measles virus. The protocol of experimental procedures was approved by the animal welfare and animal care committee of the National Institute of Infection Diseases (Tokyo, Japan).

(2) Collection of Cynomolgus CD34⁺ Cells

Cynomolgus monkeys received recombinant human (rh)SCF (50 μg/kg; Amgen) and rhG-CSF (50 μg/kg; Chugai, Tokyo, Japan) as daily subcutaneous injections for 5 days prior to blood cell collection. Peripheral blood or bone marrow cells were then collected by leukapheresis or by aspiration from iliac bones, respectively. From the harvested peripheral blood cells, the leukocyte cell fraction was obtained after red blood cell lysis with ACK buffer (155 mM NH₄CL, 10 mM KHCO₃ and 0.1 mM EDTA; Wako). Enrichment of CD34⁺ cells was performed using magnet beads conjugated with anti-human CD34 (clone 561; Dynal, Lake Success, N.Y.) that cross-reacts with cynomolgus CD34 (Shibata et al., Am J Primatol 2003, 61: 3-12). The purity of CD34⁺ cells ranged from 90 to 95% as assessed with another anti-human CD34 (clone 563; PharMingen, San Diego, Calif.) that cross-reacts with cynomolgus CD34 (Shibata et al., Am J Primatol 2003, 61: 3-12). Mean CFU enrichment was 48-fold as assessed by colony-forming progenitor assays performed before and after enrichment.

(3) Retroviral Transduction

Retroviral vector expressing SAG (a chimeric gene of human EPO receptor extracellular plus trans-membrane region and c-Mpl cytoplasmic region) (see, Example 1; Nagashima et al., J Gene Med 2004, 6: 22-31), and PLI non-expression retroviral vector containing a non-translated neo^{R} and β-gal sequences (Heim et al., Mol Ther 2000, 1: 533-44) were used. The titers of the viral supernatants used in the present Example were both 1×10⁶ particles per ml, as assessed by RNA dot-blot. CD34⁺ cells were cultured at a starting concentration of 1 to 5×10⁵ cells/ml in fresh vector supernatant of PLI or SAG with rhSCF (Amgen), rh thrombopoietin (Kirin) and rhFlt-3 ligand (Research Diagnostics) each at 100 ng/ml in dishes coated with 20 μg/cm² of RetroNectin (Takara Bio). Every 24 hr, culture medium was replaced with) fresh vector supernatant and cytokines. After 96-hr transduction, cells were washed and continued in culture (DMEM (Gibco, Rockville, Md.) containing 10% FCS (Gibco) and 100 ng/ml rhSCF alone) for two additional days in the same RetroNectin-coated dishes (Takatoku et al., J Clin Invest 2001, 108: 447-55).

(4) Intra-Bone Marrow Transplantation

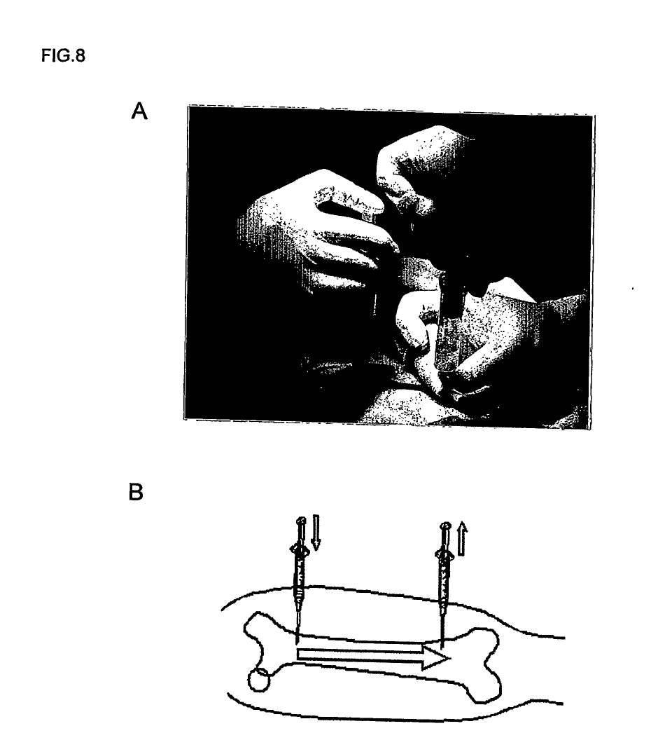

Cynomolgus monkeys were anesthetized. Two needles were inserted into both ends of the femurs or humeri (Kushida et al., Stem Cells 2002, 20: 155-62). A syringe containing 50 ml of heparin-added saline was connected to one needle and an empty syringe was connected to the other. Normal saline was irrigated gently from one syringe to the other through the marrow cavity twice (FIG. 8). Gene-modified cells were suspended in 1 ml of PBS containing 10% autologous serum, injected into the marrow cavity, and the needle holes were sealed with bone wax (Lukens, Reading, Pa.). The internal pressure in the marrow cavity during the injection procedure was measured in some animals, and carefully performed saline irrigation and iBMT without inflicting extra-pressure to the marrow cavity. No animal suffered from neutropenia, thrombocytopenia, infection or pulmonary embolism and there was no morbidity. After transplantation, rhEPO (Chugai) was subcutaneously administered to some animals at a dose of 200 IU/kg once or twice daily. Administration of cyclosporin A (Novartis, Basel, Switzerland) was started to animals a week prior to the EPO administration to prevent development of anti-human EPO antibody (Schuurmann et al., Transpl Int 2001, 14: 320-8).

(5) Clonogenic Hematopoietic Progenitor Assays

Cells were plated in a 35-mm petri-dish in 1 ml of α-minimum essential medium containing 1.2% methylcellulose (Shin-Etsu Chemicals) supplemented with 100 ng/ml rhIL-3 (PeproTech, Rocky Hill, N.J.), 100 ng/ml rh interleukin-11 (PeproTech), 100 ng/ml rhSCF (Biosource, Camarillo, Calif.), 2 U/ml rhEPO (Roche, Basel, Switzerland), 20% FCS, 1% BSA, 5×10⁻⁵ M 2-mercaptoetanol (Sigma) and antibiotics (100 U/ml penicillin and 0.1 mg/ml streptomycin). In the culture for colony formation from SAG-transduced cells, rhEPO was not added to avoid excess proliferative response of the transduced cells to EPO. After incubation for 14 days at 37° C. with 5% CO₂, colonies containing greater than 50 cells were counted using an inverted light microscope. Experiments were conducted in triplicate.

(6) Quantitative PCR

Genomic DNA was extracted using the QIAamp DNA Blood Mini Kit (Qiagen, Chatsworth, Calif.). DNA (250 ng) was amplified in triplicate with neo-specific primers for PLI (5′-TCC ATC ATG GAT GCA ATG CGG C-3′ and 5′-GAT AGA AGG CGA TGC GCT GCG AAT CG-3′) or with SAG-specific primers (5′-GAC GCT CTC CCT CAT CCT CGT-3′ and 5′-GAG GAC TTG GGG AGG ATT TCA-3′). Standards consisted of DNA extracted from an SAG- or PLI-producer cell line (with a known copy number of the proviral sequence) serially diluted with control cynomolgus genomic DNA. Negative controls consisted of DNA extracted from peripheral blood cells of naive monkeys. A β-actin-specific primer set (5′-CCT ATC AGA AAG TGG TGG CTG G-3′ and 5′-TTG GAC AGC AAG AAA GTG AGC TT-3′) was used to certify equal loading of DNA per reaction. Reactions were run using the Qiagen SYBR Green PCR Master Mix (Qiagen) on the ABI PRISM 7700 Sequence Detection System (Applied Biosystems, Foster City, Calif.) under following conditions: 50° C. for 2 min and 95° C. for 15 min, followed by 40 cycles of 94° C. for 15 sec, 62° C. for 30 sec, 72° C. for 30 sec and 83° C. for 15 sec. The quantitative PCR was certified each time to yield linear amplifications in the range of the intensity of positive control series (0.01 to 100%, correlation coefficient >0.98). For calculating the transduction efficiencies, the Ct value of the vector sequence was normalized based on the Ct value of the internal control β-actin sequence on the same sample as directed in the manufacturer's protocol. Gene marking percentages were calculated given that each provirus-positive cell contains one copy of the vector sequence.

(7) Colony PCR

Well-separated, individual colonies at day 14 were plucked into 50 μl of distilled water, digested with 20 μg/ml proteinase K (Takara Bio) at 55° C. for 1 hr followed by 99° C. for 10 min, and assessed for the SAG or non-expression PLI vector sequence by nested PCR. The outer primer sets were the same as that used in the quantitative PCR described above. Amplification conditions for the outer PCR were 95° C. for 1 min, 54° C. for 1 min, and 72° C. for 2 min with 20 cycles. The outer PCR products were purified using MicroSpin S-400 HR Columns (Amersham, Piscataway, N.J.). The inner primer set for the SAG vector was 5′-CCA CCC CTA GCC CTA AAT CTT ATG-3′ and 5′-GGT GGT TCA GCA TCC AAT AAG G-3′, and that for PLI vector was 5′-ATA C-GC TTG ATC CGG CTA CCT G-3′ and 5′-GAT ACC GTA AAG CAC GAG GAA G-3′. Amplification conditions for the inner PCR were 95° C. for 1 min, 54° C. for 1 min, and 72° C. for 2 min with 20 cycles. Simultaneous PCR for the β-actin sequence was also performed to certify DNA amplification of the sample in each colony. The primer set for β-actin was the same as that used in the quantitative PCR described above. Amplification conditions for β-actin PCR were 95° C. for 1 min, 54° C. for 1 min, and 72° C. for 2 min with 30 cycles. The final PCR products were separated on 2% agarose gels. The sizes of the products were 206, 483 and 232 bp for SAG, non-expressing PLI vector and β-actin sequences, respectively. The transduction efficiency of CFU was calculated by dividing the number of colonies positive for the vector sequence by the number positive for the β-actin sequence. Plucked methylcellulose not containing colonies served as negative controls.

(8) In Situ PCR

In situ detection of transplanted cell progeny was performed by amplifying the SAG sequence as previously reported (Haase et al., Proc Natl Acad Sci USA 1990, 87: 4971-5). Peripheral blood nucleated cells were spun down to slide glasses. The SAG-specific primer sequences were the same as those used for the quantitative PCR described above. The reaction mixture consisted of 420 μM DATP, 420 μM dCTP, 420 μM dGTP, 378 μM dTTP, 42 μM digoxigenin-labeled dUTP (Roche), 0.8 μM of each SAG primer, 4.5 mM MgCl₂, PCR buffer (Mg²⁺ free) and 4 U Takara Taq DNA polymerase (Takara Bio). PCR was performed using the PTC100 Peltier Thermal Cycler (MJ Research, Watertown, Mass.) with the following conditions: 94° C. for 1 min and 55° C. for 1 min with 15 cycles. The digoxigenin-incorporated DNA fragments were detected using the horseradish peroxidase (HRP)-conjugated rabbit F(ab′) anti-digoxigenin antibody (Dako). Slides were then stained for HRP using the vector SG Substrate Kit. Finally, slides were counterstained with the Kernechtrot that stains nucleotides, mounted in glycerol, and examined under light microscope.

(9) LAM-PCR

The LAM-PCR was performed as previously described (Schmidt et al. Nat Med 2003, 9: 463-8). The genomic-proviral junction sequence was preamplified by repeated primer extension using 0.25 pmol of vector-specific, 5′-biotinylated primer LTR1 (5′-AGC TGT TCC ATC TGT TCT TGG CCC T-3′) with Taq polymerase (2.5 U; Qiagen) from 100 ng of each sample DNA. One hundred cycles of amplification were performed with the addition of fresh Taq polymerase (2.5 U) after 50 cycles. Biotinylated extension products were selected with 200 μg of magnetic beads (Dynabeads kilobase BINDER Kit; Dynal). The samples were incubated with Klenow polymerase (2 U; Roche), dNTPs (300 μM; Pharmacia, Uppsala, Sweden), and a random hexanucleotide mixture (Roche) in a volume of 20 μL for 1 hr at 37° C. Samples were washed on the magnetic particle concentrator (Dynal) and incubated with TasI (Fermentas, Hanover, Md.) to cut the 5′-long terminal repeat-flanking genomic DNA for 1 hr at 65° C. After an additional wash step, 100 pmol of a double-stranded asymmetric linker cassette and T4 DNA ligase (6 U; New England Biolabs, Beverly, Mass.) were incubated with the beads in a volume of 10 μL at 16° C. overnight. Denaturing was performed with 5 μL of 0.1 N NaOH for 10 min at room temperature. Each ligation product was amplified with Taq polymerase (5 U; Qiagen), 25 pmol of vector-specific primer LTR2a (5′-AAC CTT GAT CTG AAC TTC TC-3′), and linker cassette primer LC1 (5′-GAC CCG GGA GAT CTG AAT TC-3′) by 35 cycles of PCR (denaturation at 95° C. for 60 sec, annealing at 60° C. for 45 sec, and extension at 72° C. for 60 sec). Of each PCR produce, 0.2% served as a template for a second, nested PCR with internal primers LTR3 (5′-TCC ATG CCT TGC AAA ATG GC-3′) and LC2 (5′-GAT CTG AAT TCA GTG GCA CAG-3′) under identical conditions. Final products were separated on a 2% agarose gel.

(10) Flow Cytometric Sorting

FSC/SSC profile (forward and side scatter) was used to sort granulocytes (purity 95%). Anti-CD3 and anti-CD20 were used to sort T-lymphocytes (purity 99%) and B-lymphocytes (purity 95%), respectively. Cells were sorted using EPICS ELITE cell sorter equipped with an argon-ion laser (Beckman Coulter, Fullerton, Calif.). Data acquisition and analysis were performed using EXPO2 software (Beckman Coulter).

(11) Cellular Immune Response Assay

Peripheral blood mononuclear cells and bone marrow stromal cells were isolated from the monkey D8058. The stromal cells were transduced with a retroviral vector carrying the PLI, SAG or human EPO receptor cDNA. The transduced stromal cells were irradiated with 4,000 cGy and used as stimulator cells. Untransduced stromal cells irradiated with 4,000 cGy served as a control. The peripheral blood mononuclear cells (responder cells, 2×10⁵/well) were cocultured with the stimulator or control cells (5×10⁴/well) in 96-well, flat-bottom plates with RPMI 1640 medium (Sigma) containing 10% fetal calf serum and 20 IU/ml rh IL-2 (Shionogi, Osaka, Japan). After 5 days of culture, the blastogenesis of responder cells was assessed. Briefly, the cells were labeled with 1 μCi/well of [methyl-3H]thymidine (Amersham) for 16 hr and harvested with an automated cell harvester (Laboratory Science, Tokyo, Japan) onto glass-fiber filters (Molecular Devices, Sunnyvale, Calif.). The incorporation of [methyl-3H]thymidine into responder cells was quantified in liquid scintillation counter (Aloka, Tokyo, Japan). All experiments were performed in triplicate.

Results

(1) Engraftment after iBMT

First, it was examined whether gene-marked CD34⁺ cells engraft after iBMT using cynomolgus macaques. Cynomolgus CD34⁺ cells were transduced with the non-expression retroviral vector PLI (containing non-translated sequence) (Heim et al., Mol Ther 2000, 1: 533-44). The transduction results are summarized in Table 2.

TABLE 2
Ex vivo transduction

| | | | | |
|---|---|---|---|---|
| | | | No. of | Fraction of |
| | Target | | infused | provirus-positive |
| Animal | cell | | CD34⁺ | CFUs in infused |
| no. | source | Vector | cells/kg | CD34⁺ cells |

Intra-bone marrow transplantation

| | | | | |
|---|---|---|---|---|
| IB3048 | bone | PLI | 4.5 × 10⁷ | 34/46 (73.9%) |
| | marrow | | | |
| IB3053 | peripheral | PLI | 8.1 × 10⁶ | 49/78 (62.8%) |
| | blood | | | |
| S9042 | peripheral | SAG | 2.6 × 10⁷ | 20/35 (57.1%) |
| | blood | | | |
| S3047 | peripheral | SAG | 8.1 × 10⁶ | 11/21 (52.4%) |
| | blood | | | |
| D8058 | peripheral | SAG | 7.8 × 10⁵ | 11/43 (25.6%) |
| | blood | PLI | 5.7 × 10⁵ | 9/42 (21.4%) |

Intravenous transplantation

| | | | | |
|---|---|---|---|---|
| V0065 | peripheral | PLI | 1.2 × 10⁷ | 3/45 (6.7%) |
| | blood | | | |
| V1007 | peripheral | PLI | 1.5 × 10⁶ | 14/41 (34.1%) |
| | blood | | | |
| S6046 | peripheral | SAG | 1.3 × 10⁵ | 3/14 (21.4%) |
| | blood | | | |

PLI: non-expression vector; and
SAG: selective amplifier gene vector

Transduced CD34⁺ cells were directly injected into the bone marrow cavity of four proximal limb bones (the femurs and humeri) after gently irrigating the cavity with saline (FIG. 8). This transplant procedure was safely preformed without pulmonary embolism or infection of bone marrow. Conditioning treatment such as irradiation was not conducted prior to transplantation. In addition, the transduced CD34⁺ cells were returned into two monkeys by a conventional transplantation method without prior conditioning.

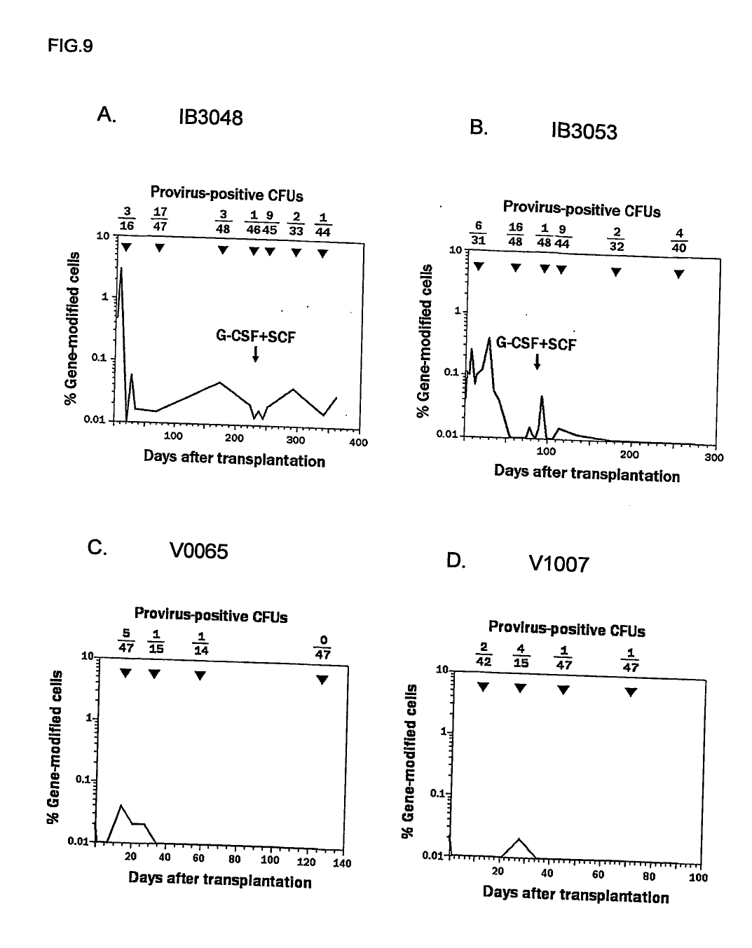

After iBMT, cells from the non-transplanted iliac marrow were plated in methylcellulose media. The resulting colonies (CFU) were examined for the provirus by PCR (FIGS. 9A and 9B). Two to 30% of colonies (overall 14.2% [74/522]) were positive for the provirus and this high marking level persisted for over one year post-transplantation. On the other hand, after the conventional intravenous transplantation, generally fewer CFU contained the provirus (overall 5.5% [15/274]) in the bone marrow (FIGS. 9C and 9D). Interestingly, the provirus in CFU from the non-transplanted marrow was detectable within two weeks after iBMT. Thus, transplanted cells relocated from a transplanted bone to another at early time points. A similar early translocation post-transplantation has also been reported in mouse syngeneic iBMT and human-mouse xeno-iBMT models (Zhong et al., Blood 2002, 100: 3521-6; Wang et al., Blood 2003, 101: 2924-31; Mazurier et al, Nat Med 2003, 9: 959-63; Yahata et al., Blood 2003, 101: 2905-13). Peripheral blood cells were also examined for the provirus by quantitative PCR (FIGS. 9A and 9B). The marking levels were, however, found to be very low (<0.1%) in the peripheral blood.

Taken together, these results suggest that transplanted cells can engraft non-conditioned recipients after iBMT, however, show minimal contribution to peripheral blood compared to myeloablated recipients. The cells stay at a resting state in bone marrow without proliferation. In an attempt to proliferate and mobilize iBMT-engrafted resting progenitor cells, G-CSF and SCF were administered for five consecutive days (Horsfall et al., Br J Haematol 2000, 109: 751-8). However, no obvious increase in the vector-containing cells was observed in the peripheral blood (FIGS. 9A and 9B).

(2) EPO-Dependent Expansion with SAG

A retroviral vector expressing an SAG that is a chimeric gene of the EPO receptor gene (extracellular and transmembrane region as a molecular switch) and the human c-Mpl gene (cytoplasmic region as a signal generator) was constructed (see, Example 1; Nagashima et al., J Gene Med 2004, 6: 22-31). Cells genetically engineered to express this SAG will proliferate in an EPO-dependent manner. Cynomolgus CD34⁺ cells were transduced with the SAG retroviral vector and introduced into non-conditioned autologous recipient by iBMT (Table 2). In vivo results after transplantation is summarized in Table 3.

TABLE 3
In vivo expansion with SAG after iBMT

| | EPO treatment | | | Marked leukocytes (%)* | |
|---|---|---|---|---|---|
| | | Period | | Basal marking | Peak marking after |
| Animal | Treatment | (Days | | before | treatment |
| no. | course no. | post-transplant) | Dosage | treatment | (Day post-transplant) |
| S9042 | 1 | 1-40 | 200 IU/kg | N.A. | 7.36% (Day 105) |
| | | | once daily | | |
| | | 41-100 | 200 IU/kg | | |
| | | | twice daily | | |
| | 2 | 132-210 | 200 IU/kg | 0.02% | 7.72% (Day 188) |
| | | | twice daily | | |
| | 3 | 246-367 | 200 IU/kg | 0.41% | 8.90% (Day 348) |
| | | | twice daily | | |
| S3047 | 1 | 75-134 | 200 IU/kg | 0.01% | 0.23% (Day 145) |
| | | | once daily | | |
| | | 135-166 | 200 IU/kg | | |
| | | | twice daily | | |
| | 2 | 210-289 | 200 IU/kg | 0.02% | 0.00% (Day 289) |
| | | | twice daily | | |
| D8058 | 1 | 1-86 | 200 IU/kg | N.A. | 2.30% (Day 14) |
| | | | twice daily | | |
| S6046 | 1 | 1-50 | 200 IU/kg | N.A. | Less than 0.01% |
| | | | twice daily | | (Day 49) |

*As assessed by quantitative PCR (see Material and Methods)
N.A.: not applicable.

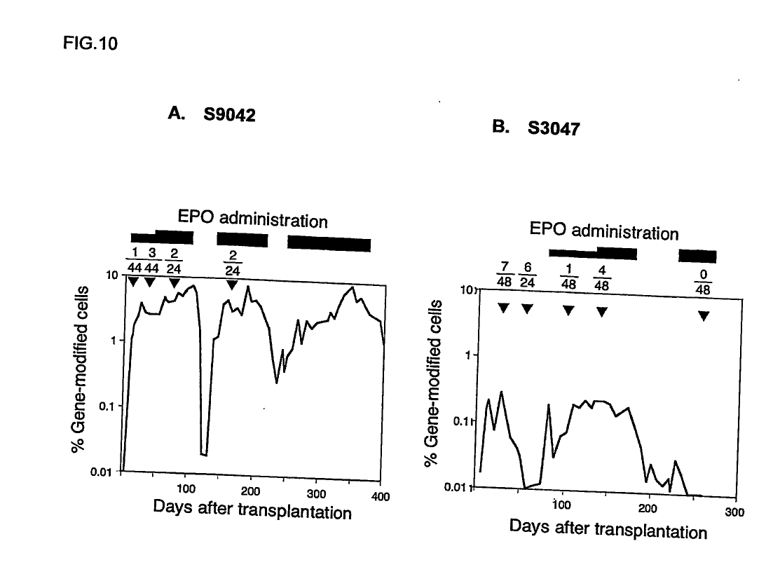

In one animal (FIG. 10A), EPO administration triggered a striking elevation in marking levels (7.4% at day 105 post-transplantation) in the peripheral blood. The levels of marking in the periphery stayed high for the duration of EPO administration. After cessation of EPO, the level fell down to <0.1%. Resumption of EPO administration produced a similar elevation in the marking levels. The third EPO administration again resulted in increased marking levels to 8.9% at day 348 post-transplantation. EPO administration was associated with a mild increase in hematocrit (up to 63.5%), which was manageable by occasional phlebotomy. No other adverse effects were observed.

In another animal (FIG. 10B), the SAG-transduced cells increased following transplantation even without exogenous EPO administration. The increase may have been due to increased endogenous EPO elevation resulting from anemia present in the second animal. Overall marking fell with resolution of the anemia. Following resolution, EPO was administered, resulting in an increase in marking levels by more than 20-fold. Marking levels declined to the basal level after discontinuation of EPO. A second attempt to increase marking levels failed, with clearance of SAG-positive cells from the periphery within a month after the second administration, most likely due to cellular immune responses to the xenogeneic SAG (see below).

Expansion of SAG-transduced cells was seen in three lineages; granulocytes, B- and T-lymphocytes. The c-Mpl signal generated by SAG may work even in lymphocytes. In fact, B-lymphocytes had been shown to increase by the activated c-Mpl in a canine transplantation model (Neff et al., Blood 2002, 100: 2026-31). The expansion was transient, similarly with other chimeric genes containing c-Mpl as a signal generator (Neff et al., Blood 2002, 100: 2026-31), although basal marking levels seemed to gradually increase after repeated EPO administrations as shown in FIG. 10A. The method largely results in the selection of transduced cells not al the level of HSCs, but within the differentiated progeny of transduced HSCs.

(3) Multilineage and Polyclonal Expansion

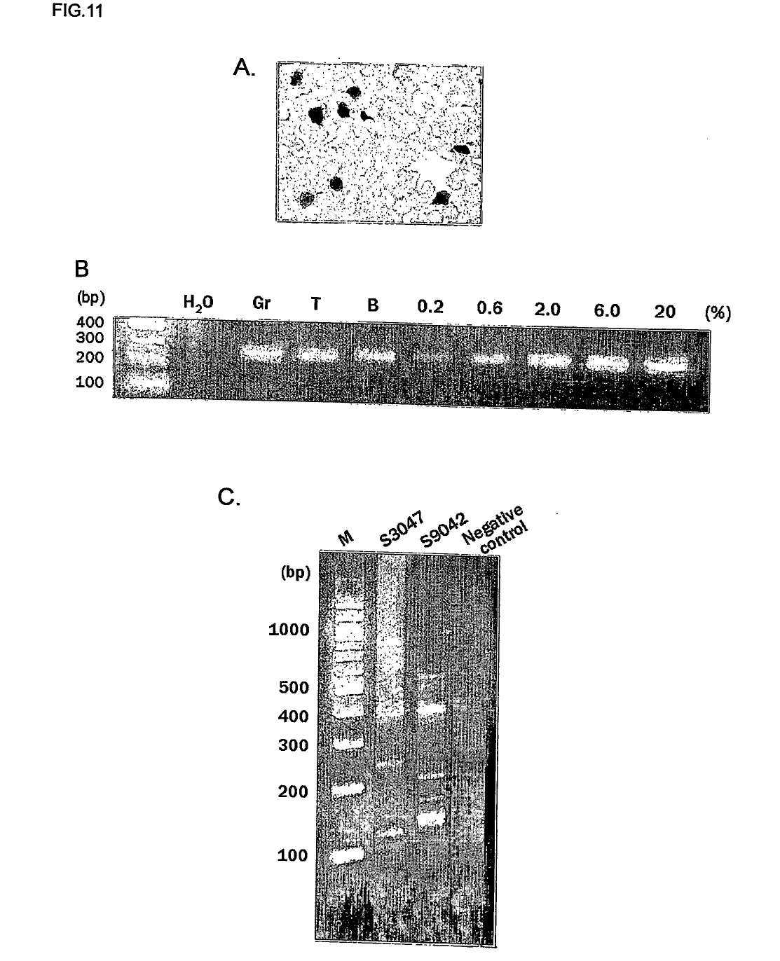

In situ PCR for the proviral sequence showed many transduced cells in the peripheral blood taken from the animal S9042 receiving EPO at day 89 post-transplantation (FIG. 11A). Granulocytes, T- and B-lymphocytes sorted from the peripheral blood of this animal at day 91 post-transplantation were subjected to semi-quantitative PCR for the provirus. The provirus-containing fraction in granulocytes was 6%, and that in B- and T-lymphocytes was 2% (FIG. 11B). This indicates the occurrence of multilineage expansion. The persistence of marked, short-lived granulocytes for long term is also another evidence for the successful engraftment of gene-modified HSCs after iBMT. The integration site analysis using the linear amplification mediated (LAM)-PCR method (Schmidt et al., Nat Med 2003, 9: 463-8) indicates that the expansion of transduced cells in response to EPO was polyclonal, not mono- or oligo-clonal (FIG. 11C).

(4) Dual Marking Study

Next, the effect of the SAG vector was compared to a non-SAG vector within, rather than between, individual animals. Cytokine-mobilized peripheral blood CD34⁺ cells were harvested and split into two equal aliquots. One aliquot was transduced with the SAG vector and the other with the control non-expression vector (PLI). Both aliquots were mixed and returned by iBMT without marrow conditioning. The animal received EPO from the day after transplantation and in vivo marking levels derived from the two populations were examined by quantitative PCR.

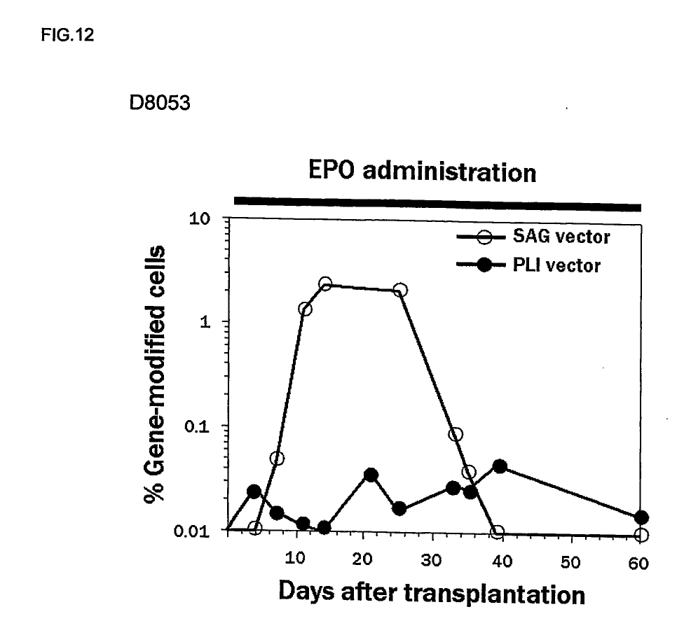

Cells containing the SAG vector increased by two logs in the peripheral blood in response to EPO, although cells containing the non-expression vector remained at low levels (FIG. 12). However, SAG-containing cells were rapidly cleared within 1 month post-transplantation from the periphery and overall SAG-vector marking level became even lower than that from the non-expression vector-marked fraction. Since cyclosporin A was concomitantly administered to prevent immune responses to human EPO, human EPO concentrations were maintained within an effective range. Thus, it is unlikely that the clearance of xenogeneic human EPO due to immune responses turned off the molecular switch of SAG, resulting in the decrease in SAG-transduced cells.

(5) Immune Responses

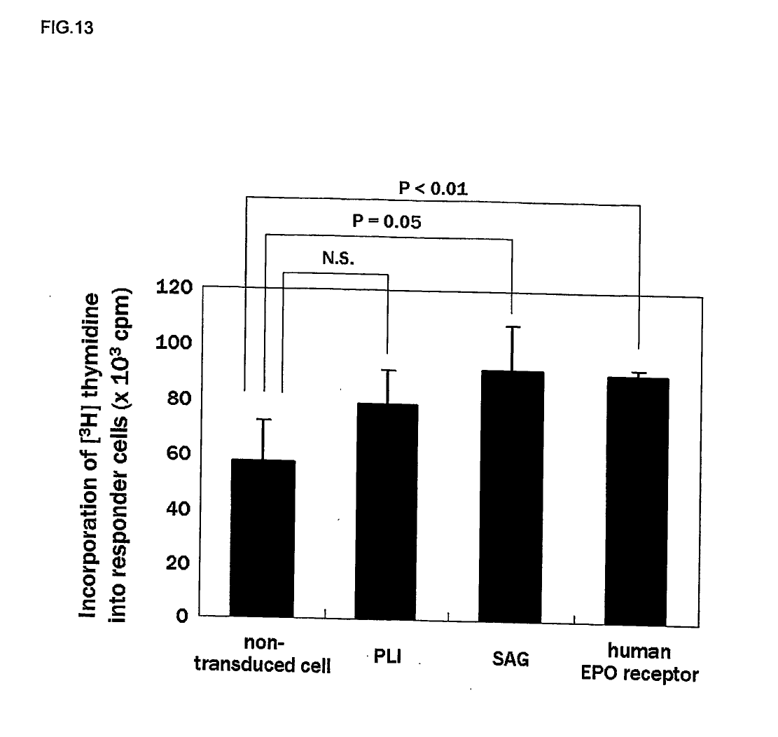

The current SAG is a chimeric gene of human origin (the human EPO receptor and human c-Mpl). Peripheral lymphocytes from the animal receiving both SAG and non-expressing PLI (D8058, FIG. 12) was collected at day 169 post-transplantation and examined whether the lymphocytes responded to the xenogeneic SAG in vitro (FIG. 13). The response to SAG-transduced target cells was stronger than that to non-transduced target cells (p=0.05), while the response to PLI-transduced target cells did not significantly differ from that to non-transduced target cells (p=0.13). The cellular immune response is, therefore, the most likely reason for the clearance of SAG-transduced cells in this animal. This is not novel, but it has been reported that immune responses against transgene products recognized as foreign can indeed be a major obstacle to long-term persistence of gene-modified cells in vivo (Heim et al., Mol Ther 2000, 1: 533-44; Riddle et al., Nat Med 1996, 2: 216-23; Rosenzweig et al., Blood 2001, 97: 1951-9). In the human clinical setting, however, immune responses should not occur against SAG, because SAG is made of human genes.

(6) In Vivo Effect of the Combination of SAG and iBMT

The gene transduction efficiency was examined in peripheral blood 26 days post-transplantation by PCR. When the SAG transduced cells were transplanted intravenously, compared to the high efficiency obtained by iBMT dropped to less than 0.01%, i.e., below the detection level (Table 3).

Even without bone marrow conditioning, SAG transduced cells are detected at a high efficiency of 8% in the peripheral blood through iBMT. However, when the cells are transplanted by the IV method, the transduction efficiency in the peripheral blood becomes extremely low. Therefore, to achieve a high efficiency for gene transduced hematopoietic stem cells without bone marrow conditioning, it is indispensable to combine SAG and iBMT.

### INDUSTRIAL APPLICABILITY

In vivo expansion of gene-modified cells is a promising approach in the field of HSC gene therapy. According to the present invention, the combination of SAG and iBMT was demonstrated to realize a high marking level for a long period. Therefore, the present method for transplanting lymphohematopoietic cells into mammals is particularly suited for HSC gene therapy.

Furthermore, a new generation SAG that utilizes the ligand-binding domain of EPOR was provided by the present invention. This new generation SAG was demonstrated to induce more rapid and potent proliferation of cells than the foregoing steroid-driven SAGs. Moreover, the ligand (EPO) used for this SAG is suggested to be safer for administration in vivo than those used for the steroid-driven SAGs. Therefore, the new generation SAG of the present invention is expected to contribute to the clinical application of not only HSC gene therapy but also adult stem cell therapy.

While the invention has been described in detail and with reference to specific embodiments thereof, it will be apparent to one skilled in the art that various changes and modifications can be made therein without departing from the spirit and scope of the invention.

1. A method for transplanting lymphohematopoietic cells into a mammal, which comprises the step of injecting cells into a bone marrow cavity, and wherein the cells have an exogenous gene encoding a receptor that induces cell proliferation in response to ligand binding.

2. The method of claim 1, which lacks the step of marrow conditioning before injection of the cells.

3. The method of claim 1, wherein the exogenous gene has been introduced into the cell using a viral vector.

4. The method of claim 1, wherein the receptor is a chimeric protein having (a) an extracellular domain of a receptor that dimerizes the chimeric protein in response to ligand binding, and (b) a growth signal generator that induces cell proliferation in response to the dimerization.

5. The method of claim 1, wherein the receptor has a cytoplasmic domain of a hematopoietic cytokine receptor.

6. The method of claim 1, wherein the receptor has a cytoplasmic domain of a thrombopoietin (TPO) receptor or a granulocyte colony-stimulating factor (G-CSF) receptor.

7. The method of claim 1, wherein the receptor has an extracellular domain of an erythropoietin (EPO) receptor.

8. The method of claim 1, wherein the cell is a pluripotent stem cell.

9. The method of claim 1, wherein the mammal is a primate.

10. The method of claim 1, wherein the method comprises the step of administering a ligand of the receptor into the mammal.

11. The method of claim 1, wherein the cell comprises a vector having a therapeutic gene.

12. A bone marrow transplant comprising (a) lymphohematopoietic cells having an exogenous gene encoding a receptor that induces cell proliferation in response to ligand binding, and (b) a pharmaceutically acceptable carrier.

13. A kit for transplanting lymphohematopoietic cells into a mammal, which comprises (a) a vector encoding a receptor that induces cell proliferation in response to ligand binding, and (b) a recording medium describing the use of the vector and lymphohematopoietic cells introduced with the vector for injection into the bone marrow cavity.

14. A gene encoding a fusion protein comprising (a) a ligand-binding domain of erythropoietin (EPO) receptor, and (b) a growth signal generator that imparts proliferation activity to a cell upon the binding of a ligand.

15. The gene of claim 14, wherein the growth signal generator is a cytoplasmic domain derived from the granulocyte colony-stimulating factor (G-CSF) receptor or thrombopoietin (TPO) receptor.


SEQUENCE LISTING (25 sequences)

SEQ ID NO: 1 - DNA, 29 bp, Artificial Sequence
  an artificially synthesized primer sequence
aaggatccaaacgcagaggaaagaagact

SEQ ID NO: 2 - DNA, 26 bp, Artificial Sequence
  an artificially synthesized primer sequence
aagtcgacctagaaacccccttgttc

SEQ ID NO: 3 - DNA, 26 bp, Artificial Sequence
  an artificially synthesized primer sequence
aaggatccaggtggcagtttcctgca

SEQ ID NO: 4 - DNA, 26 bp, Artificial Sequence
  an artificially synthesized primer sequence
cggtcgactcaaggctgctgccaata

SEQ ID NO: 5 - DNA, 23 bp, Artificial Sequence
  an artificially synthesized primer sequence
ctcggccggcaacggcgcaggga

SEQ ID NO: 6 - DNA, 26 bp, Artificial Sequence
  an artificially synthesized primer sequence
aaggatcccagcagcgcgagcacggt

SEQ ID NO: 7 - DNA, 22 bp, Artificial Sequence
  an artificially synthesized primer sequence
cgtccaggagcgcaccatcttc

SEQ ID NO: 8 - DNA, 21 bp, Artificial Sequence
  an artificially synthesized primer sequence
agtccgccctgagcaaagacc

SEQ ID NO: 9 - DNA, 24 bp, Artificial Sequence
  an artificially synthesized primer sequence
cattgtcatggactctggcgacgg

SEQ ID NO: 10 - DNA, 24 bp, Artificial Sequence
  an artificially synthesized primer sequence
catctcctgctcgaagtctagggc

SEQ ID NO: 11 - DNA, 22 bp, Artificial Sequence
  an artificially synthesized primer sequence
tccatcatggatgcaatgcggc

SEQ ID NO: 12 - DNA, 26 bp, Artificial Sequence
  an artificially synthesized primer sequence
gatagaaggcgatgcgctgcgaatcg

SEQ ID NO: 13 - DNA, 21 bp, Artificial Sequence
  an artificially synthesized primer sequence
gacgctctccctcatcctcgt

SEQ ID NO: 14 - DNA, 21 bp, Artificial Sequence
  an artificially synthesized primer sequence
gaggacttggggaggatttca

SEQ ID NO: 15 - DNA, 22 bp, Artificial Sequence
  an artificially synthesized primer sequence
cctatcagaaagtggtggctgg

SEQ ID NO: 16 - DNA, 23 bp, Artificial Sequence
  an artificially synthesized primer sequence
ttggacagcaagaaagtgagctt

SEQ ID NO: 17 - DNA, 24 bp, Artificial Sequence
  an artificially synthesized primer sequence
ccacccctagccctaaatcttatg

SEQ ID NO: 18 - DNA, 22 bp, Artificial Sequence
  an artificially synthesized primer sequence
ggtggttcagcatccaataagg

SEQ ID NO: 19 - DNA, 22 bp, Artificial Sequence
  an artificially synthesized primer sequence
atacgcttgatccggctacctg

SEQ ID NO: 20 - DNA, 22 bp, Artificial Sequence
  an artificially synthesized primer sequence
gataccgtaaagcacgaggaag

SEQ ID NO: 21 - DNA, 25 bp, Artificial Sequence
  an artificially synthesized primer sequence
agctgttccatctgttcttggccct

SEQ ID NO: 22 - DNA, 20 bp, Artificial Sequence
  an artificially synthesized primer sequence
aaccttgatctgaacttctc

SEQ ID NO: 23 - DNA, 20 bp, Artificial Sequence
  an artificially synthesized primer sequence
gacccgggagatctgaattc

SEQ ID NO: 24 - DNA, 20 bp, Artificial Sequence
  an artificially synthesized primer sequence
tccatgccttgcaaaatggc

SEQ ID NO: 25 - DNA, 21 bp, Artificial Sequence
  an artificially synthesized primer sequence
gatctgaattcagtggcacag